(19) United States
(12) Patent Application Publication
(10) Pub. No.: US 2021/0193290 A1
(43) Pub. Date: Jun. 24, 2021

(54) SYSTEM AND COMPUTER PROGRAM FOR ANALYZING AND MANAGING FITNESS AND NUTRITIONAL WELLNESS

(71) Applicant: EVA LLC, Southlake, TX (US)

(72) Inventor: James J. Mahoney, Southlake, TX (US)

(73) Assignee: EVA LLC, Southlake, TX (US)

(21) Appl. No.: 17/075,854

(22) Filed: Oct. 21, 2020

Related U.S. Application Data
(63) Continuation-in-part of application No. 15/844,606, filed on Dec. 17, 2017.
Application No. 15/844,606 is a continuation-in-part of application No. 13/933,277, filed on Jul. 2, 2013, now abandoned.

(51) Int. Cl. G16H 20/30; H04L 29/08; G16H 10/60; G16H 20/60; G16H 10/20; A61B 5/00

(52) U.S. Cl. CPC G16H 20/30; H04L 67/1097; G16H 10/60; H04L 67/12; G16H 10/20; A61B 5/743; G16H 20/60

(57) ABSTRACT

A system and computer program product for analyzing and managing fitness and nutritional wellness. The system and computer program product include receiving configuration and setup information, receiving and storing patient information in the database, correlating the responses based on the predefined correlation rules to a plurality of functional scores, creating a health and wellness graphical representation of a patient based on the functional scores, and displaying the health and wellness graphical representation within the predefined thresholds to the patient. The configuration and setup information includes configuring predefined fitness and wellness questions derived from validated questionnaires in a plurality of subspecialty fields, and predefined correlation rules associated with the fitness and wellness questions. The patient information includes responses to the predefined fitness and wellness questions.

### CROSS REFERENCE TO RELATED APPLICATIONS

The present application claims priority to U.S. non-provisional patent application Ser. No. 15/844,606, entitled “System and Computer Program for Analyzing and Managing Fitness and Nutritional Wellness”, filed in the U.S. Patent and Trademark Office on Dec. 17, 2017, which claims priority to U.S. non-provisional patent application Ser. No. 13/933,277, entitled “System and Computer Program for Analyzing and Managing Fitness and Nutritional Wellness”, filed in the U.S. Patent and Trademark Office on Jul. 2, 2013, having at least one common inventor as the present document and hereby incorporated by reference.

### BACKGROUND OF THE INVENTION

### Field of the Invention

The present invention is generally related to systems for fitness and nutritional wellness, and more particularly to a system and computer program for analyzing and managing fitness and nutritional wellness.

### Discussion of the Background

In the 1950's, pioneers in functional medicine began research in what was called molecular nutrition. These pioneers developed concepts of biochemical individuality and biomolecular psychiatry that investigated the integral relationships of nutrition and physiology.

Unfortunately today, most contemporary health care practitioners have little formal education in functional medicine and molecular or clinical nutrition. As a result, too few doctors recognize the connection between faulty nutrition and clinical disorders in their patients.

Additionally, the process of addressing the relationship between nutrition and clinical disorders is burdensome as a result of the labor and time intensive tasks of evaluation, integration and implementation. While causal relationships between metabolic dysfunction, biochemical individuality and age-related diseases are now scientifically recognized and validated as being closely tied to lifestyle, environmental factors, and diets that provide inappropriate macro and micro nutritional constituents, the sheer volume of currently available peer-reviewed science has outstripped the typical consumer as well as physician's ability to implement lifestyle, fitness, dietary and nutritional modifications based on relevant, currently available scientific evidence.

As a result, incongruent advice and resultant confusion permeates the health, nutrition and preventive care industries regarding what, when and how to implement proper age and gender specific diet, nutrition, exercise and remedial lifestyle recommendations. Moreover, the health care industry is inundated with conflicting opinions engendering inconsistent advice regarding diet, health, lifestyle, exercise, and disease prevention strategies for various populations.

The nutritional industry offers a widely divergent array of products that when utilized, separately or in combination, may or may not be beneficial to an individual based on their age, gender, genetic, environmental and lifestyle factors, formulation, and method of administration. Furthermore, many consumers are not aware of the potential overlap or superfluous combinations of ingredients in their supplement programs that may combine to provide nutritional ingredient amounts in excess of what is clinically relevant, validated, or necessary for healthy support.

Most consumers do not discuss nutritional support products with their primary health care providers and therefore, miss the positive synergy that could occur as the result of a properly implemented, integrated nutritional/medical plan.

Beyond these factors, efficacious management of an individual's healthcare needs should include remedial measures associated with functional medicine. Current peer-reviewed science validates the following four factors that make a nutritional approach to functional medicine a foundational aspect of healthcare today: (i) nutrition is an environmental factor that influences gene expression and phenotype in each individual, (ii) nutrients act as important biological response modifiers and control/regulate function of tissues, glands, organs and systems throughout the body, (iii) the molecular environment of the body depends on the interaction of an individual's genes with macronutrients, micronutrients, and conditionally essential nutrients, and (iv) diseases such as cardiac disease, adult-onset diabetes, arthritis, digestive disorders, loss of cognitive function and many forms of cancer are often the result of an amalgamation of multiple factors including nutritional under-consumption, poor dietary, lifestyle, fitness and environmental choices.

Thus, there currently exist deficiencies in analyzing and managing fitness and nutritional wellness.

### SUMMARY OF THE INVENTION

Accordingly, one aspect of the present invention is to provide a system for analyzing and managing fitness and nutritional wellness. The system includes a plurality of remote computers, a central server, a network interface in communication with the central server and the plurality of remote computers over a network, and a shared database in communication with the central server. The network interface is configured to receive patient information. The central server is configured to receive configuration and setup information, receive and store patient information in the database, correlate the responses based on the predefined correlation rules to a plurality of functional scores, create a health and wellness graphical representation of a patient based on the functional scores, and display the health and wellness graphical representation within the predefined thresholds to the patient. The configuration and setup information includes configuring predefined fitness and wellness questions derived from validated questionnaires in a plurality of subspecialty fields, and predefined correlation rules associated with the fitness and wellness questions. The patient information includes responses to the predefined fitness and wellness questions.

Yet another aspect of the present invention is to provide a computer program product embodied on a non-transitory computer readable medium for analyzing and managing fitness and nutritional wellness. The computer program is implemented by one or more processors executing processor instructions. The computer program product includes a first computer code for receiving configuration and setup information, a second computer code for receiving and storing patient information in the database, a third computer code for correlating the responses based on the predefined correlation rules to a plurality of functional scores, a fourth computer code for creating a health and wellness graphical representation of a patient based on the functional scores, and a fifth computer code for displaying the health and wellness graphical representation within the predefined thresholds to the patient. The configuration and setup information includes configuring predefined fitness and wellness questions, and predefined correlation rules associated with the fitness and wellness questions. The patient information includes responses to the predefined fitness and wellness questions.

### BRIEF DESCRIPTION OF THE DRAWINGS

A more complete appreciation of the present invention and many of the attendant advantages thereof will be readily obtained as the same becomes better understood by reference to the following detailed description when considered in conjunction with the accompanying drawings, wherein:

FIGS. 1A-1B are block diagrams illustrating a system for analyzing and managing fitness and nutritional wellness in accordance with an embodiment of the present invention;

FIGS. 2A-2E are flow charts illustrating a method for analyzing and managing fitness and nutritional wellness in accordance with an embodiment of the present invention;

FIGS. 3A-3B illustrate an exemplary user interface for graphically presenting fitness and nutritional wellness information in accordance with an embodiment of the present invention;

FIGS. 3C-3E illustrate an exemplary user interface for graphically presenting fitness and nutritional wellness information in accordance with an alternate embodiment of the present invention; and

FIGS. 4A-4V illustrate an exemplary user interface and computer program for analyzing and managing fitness and nutritional wellness in accordance with an embodiment of the present invention.

### DETAILED DESCRIPTION THE PREFERRED EMBODIMENTS

Referring now to the drawings, wherein like reference numerals designate identical or corresponding parts throughout the several views, preferred embodiments of the present invention are described.

According to one embodiment, predefined thresholds and an easy to use user interface is utilized to reduce prejudice or inaccuracy. The intuitive interface is configurable to conform to a user's practice style or modularity. The system allows substantially all of the practice preference to be configured, including without limitation how the questionnaire is presented.

The present invention utilizes a composite score resulting from symptom interview in combination with objective data. According to one embodiment, the objective data is received from a HL7 bridge. However, it is understood that objective data may be received from other sources within the scope of the present invention. The resulting data is translated into a diagnostic image generator for the purpose of creating a human-machine interface. The human-machine interface of the present invention generates a graphical representation of the many layers of complex symptomatic and objective data. Thereby, the many layers of complex symptomatic and objective data can be analyzed graphically by the human eye. According to one embodiment, the present invention maintains historical information over a prescribed period of time for a patient. This historical information is used by the present invention to generate images for evaluation over a prescribed time period. Instead of reading text, medical images and laboratory reports to come to a valid medical decision the present invention provides a computer generated image that presents the interpreted data in a graphic context for rapid acquisition of highly predictive health and wellness conclusions.

In a sense, a picture that is worth many thousands of words. The system applies an interpretive algorithm to all classes of data to build an image that summarizes all of the data and diagnostic probabilities. This diagnostic image is presented to the human eye similarly to a bar code or UPC symbol would be to an electronic “reader.” The subtle inferences and nuances of the human mind are employed to create the final health and wellness conclusions.

The system utilizes multiple questionnaires grouped into different health and wellness categories. However, the questionnaire categories are overlaid to impact global rather than just specific diagnoses. The questionnaires include reduced diagnostic choices to improve analysis and minimize diagnostic errors. Each of the responses to the questionnaires have a configurable numerical score. The questions presented and the numerical scores associated with the responses are configurable. A rules-based engine analyzes the questionnaire results and based on the configurable rules, arrives at an overall numerical score for each category. Reports, graphical representations and recommendations are then provided to the user. Historical information is maintained so that trend analysis can be performed. A sequential graphic improvement score and/or trend chart may be presented to the user showing the user's improvement over time. The system identifies risk factors and may be configured to send an automated warning communication to the user via email if a risk threshold is exceeded.

The system may be configured to assist the doctor in screening and/or prescreening patients. The doctor can upload and/or add comprehensive doctor's notes for a treatment plan.

The system includes a supplement store linked to outputs from single question to complex functional output.

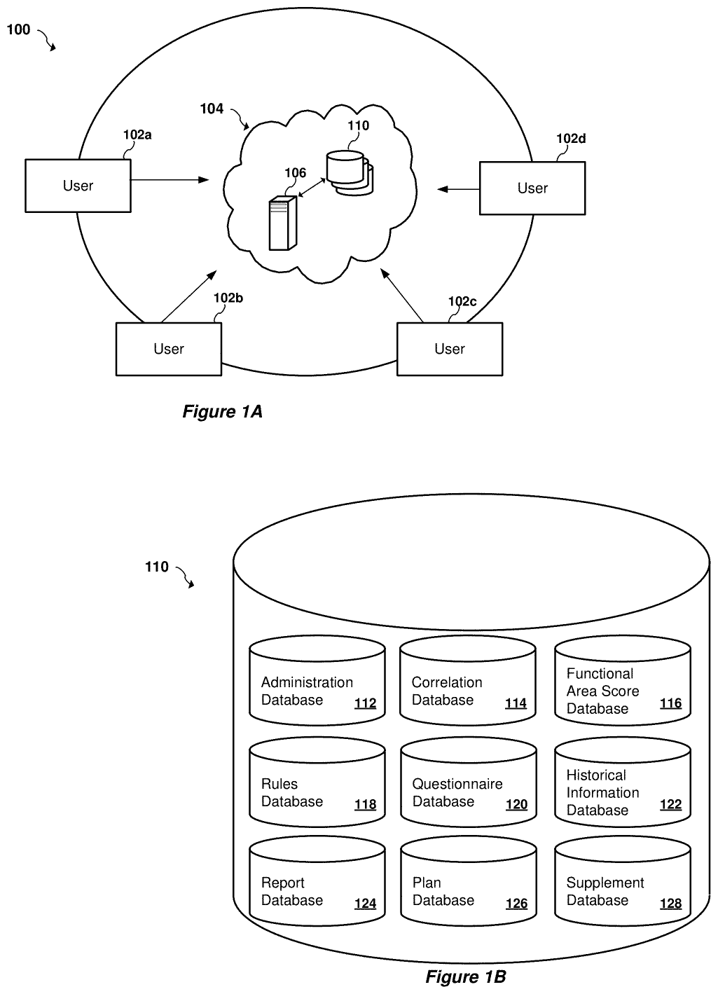

Referring to FIGS. 1A-1B, block diagrams illustrating a system 100 for analyzing and managing fitness and nutritional wellness in accordance with an embodiment of the present invention is shown. The system 100 includes one or more computers and/or servers 106 in communication with one or more databases 110. According to one embodiment, the one or more databases 110 include without limitation an administration database 112, a correlation database 114, a functional area score database 116, a rules database 118, a questionnaire database 120, a historical information database 122, a report database 124, a plan database 126, and a supplement database 128. It is understood, however, that other databases and/or combinations thereof are possible within the scope of the present invention. According to one embodiment, one or more users (102a-102d) using an interface device may access the system 100 via a network 104. The interface device may include without limitation a computer, laptop, PDA, smart phone, server, and the like. The network may include without limitation the Internet, Intranet, LAN or WAN, and the like. The one or more users (102a-102d) may utilize a web browser or other software to communicate with the system 100. According to an alternate embodiment, the system 100 is a standalone version loaded on a computer of the one or more users (102a-102d), which may or may not include a network.

The system 100 receives input data from various sources, including without limitation input directly from an interface device (not shown) by a user, a clinician, or a laboratory. A system 100 may be accessed via a secure website by the interface device through a socket of a secure encrypted server. Once the website is accessed, the user may be presented with a set of dynamically changing questions that will elicit particular input data.

The types of input data that may be received into the system 100 include but are not limited to the types of described in Table 1 below.

TABLE 1
EXEMPLARY INPUT DATA

| Type | Description |
|---|---|
| Demographics | Information that distinguishes a |
| | user including data points like age, |
| | weight, height, gender etc. |
| Family History | Information pertaining to historical |
| | family environmental, medical, |
| | physiological, and/or psychological |
| | conditions. |
| Personal History | Information pertaining to historical, |
| | personal, environmental, medical, |
| | physiological, and/or psychological |
| | conditions. |
| Social History | Information regarding specific |
| | socio-environmental, sociological, and |
| | various social conditions that a person |
| | currently is, or has been exposed |
| | to in the past. |
| Current | Information that pertains to the |
| Supplements | supplements that are currently being |
| Utilized | utilized by the user. |
| Lifestyle | Information that pertains to a person's |
| | habitual lifestyle patterns |
| | including, but not limited to habitual |
| | thoughts and mental outlook, |
| | exercise, fitness, eating habits, |
| | smoking habits, and alcohol |
| | consumption etc. |
| Body's Red Flags | Information regarding a person's |
| | perceived and objective measures of |
| | well-being including emotional, global |
| | and specific physical, and |
| | global and specific mental measures. |
| Oriental | Physical characteristics and behavioral |
| Medicine | patterns suggest specific organ |
| Biopsychotype | system dysfunction and failure along |
| | predictable lines according to |
| | well-documented principles of Eastern |
| | medicine. The system identifies |
| | these patterns and directs users and |
| | physicians to scrutinize these |
| | specific organ systems for their |
| | possible contribution to health |
| | diminishment. |

In addition to input data, the system 100 is capable of utilizing input data from clinical examination procedures and laboratory studies. Clinical and laboratory data also may be input through a secure socket connection of the secure server or through an electronic data interface with a laboratory or clinic. Clinical data is defined as any information gathered by all means during a direct encounter with the client present. Laboratory data includes any process of collecting and assessing biochemical metabolites, markers, laboratory values, and information generated from analytical data including, but not limited to: salivary or pH tests; blood, blood plasma, or blood spot tests; urinary tests; stool samples; and caliper readings.

Additionally, the system 100 is capable of utilizing input data from biometric measurement devices. Biometric data may include, but is not limited to: EKG data; EEG data; biometric scan data, digitized image data, electromagnetic feedback reader data, and electronic energy flow detection device data. At least a portion of the input data is entered into a correlation database 114 of an expert system engine.

The output data may be utilized to improve quality of life and genetic expression. A goal of the system is to modify the functional genetic expression evidenced through improved laboratory parameters, sense of well-being, and objective physical and behavioral parameters. Output data may include, but is not limited to, the output data shown in Table 2 below.

TABLE 2
EXEMPLARY OUTPUT DATA

| Type | Description |
|---|---|
| Lifestyle Plan | Customized lifestyle tips shown to |
| | address pertinent issues the user is |
| | facing. |
| Precision | Stretching, cardiovascular, and |
| Fitness Plan | resistance training plans specifically |
| | designed according to the users |
| | exercise history, ability, and goals. |
| | Maximal and training heart rate, |
| | duration, method, type of exercise, and |
| | precise rest periods are among the |
| | essential components that produce |
| | benefit in this plan. |
| Precision | An eating plan that determines the |
| Eating Plan | particular carbohydrate to protein |
| | ratio for the user determined by |
| | calculation of specific inputs the user |
| | provided the system. The eating plan |
| | uses different foods and feeding |
| | schedules to maximize endogenous |
| | homeostatic control on multiple |
| | levels. |
| Nutrition Plan | An individualized compilation of |
| | foods to eat that will benefit the |
| | individual as well as a compilation |
| | of foods to avoid due to their |
| | negative impact on digestion, |
| | physiology, symptoms or |
| | digestive/allergic properties is a |
| | significant contributor to the effect of |
| | the plan. Blood type, allergy, |
| | underlying functional challenges and |
| | food preferences are integrated |
| | into the system. |
| Nutritional | Function-enhancing and supporting |
| Supplements | nutrients and agents targeted to a |
| Plan | unique profile (individually and |
| | combined in defined proportions). |
| Nutritional | The ingredients in each supplement |
| Supplement | that the user is currently consuming |
| Inventory | is provided. These ingredients are |
| Data | compared with independently lab |
| | certified or otherwise third-party |
| | verified supplement data. The data |
| | provides specific information allowing |
| | the user to make an informed |
| | decision regarding the continuation |
| | of specific supplements the user is |
| | currently taking, or make an informed |
| | decision regarding the |
| | discontinuation of specific supplements |
| | that are no longer necessary, as |
| | well as verify the importance of |
| | additional supplements that may be |
| | added to establish or more thoroughly |
| | complete the clinical and peer- |
| | reviewed observations of the system. |
| Doctor's | Information includes additional |
| Suggestions | supplements, lab tests, and therapies |
| | designed to give the user's physician |
| | a variety of doctor assisted |
| | remedies or methodologies to more |
| | accurately address the user's needs. |
| Drugs and | Drugs and other pharmaceutical |
| Pharmaceutical | preparations. |
| Preparations | |
| Biologicals | Vaccines and sera. |
| Gene modifying | Treatments which modify genes. |
| treatments | |
| Therapies | Various physical, emotional, |
| | energetic, and mental therapies |

A trend represents measurable or perceived changes in the user after implementing one or more plans or utilizing the information produced in output data to initiate healthful changes in indicated areas. A trend may include areas involving e.g., disease avoidance, changes in the body's red flags, and optimal subjective health are entered into a second round of input data provided by the user. That is, the trend is not considered until the user enters input data into the process flow system 100 a second time.

The new input data corresponding to the trend is stored in a historical information database 122. Output data generated by the expert system engine is also stored in the historical information database 122. The historical information database 122 allows the trends and output data to be stored and analyzed.

A user has the ability to utilize the system 100 on a regular basis reentering input data and generating a new set of output data containing information specific to their current health situation. The data may be stored and trended allowing the process to be repeated, but with a slightly different focus and input data.

The system 100 includes a number of databases, tools, rules engines, and content generators. The system 100 includes a correlation database 114 that stores all of the input data. As previously described, the input data can be generated by the user, by a clinical examination, a laboratory study, or directly from biometric measurement devices.

The system 100 further includes a rule creation tool, which may be a simple graphic user interface to allow an expert system programmer or administrator to create a correlation rule. Each correlation rule created is capable of giving a positive, negative, or neutral value to the input data for a particular functional area. A functional area is a monitored health factor related to the user's body chemistry, brain function, and immune system for example. More specifically, a functional area may be related to the user's thyroid performance, anxiety level, and allergies. For instance, if the user identifies a family history of cardiac disease as well as a lifestyle comprised of little exercise and high fat foods, the correlation rule will weigh these combined factors as highly negative, and factor in the remainder of the input data to generate a functional area score for cardiac health. The functional area score is simply a numerical value given to the health factor after the correlation rule is applied to the input data. A highly negative functional area score for cardiac health may also impact the functional area score of another health factor and vice versa.

A rules database 118 stores the correlation rules that relate to functional areas. The correlation rules give negative and positive weights to input data that is relevant to a particular functional area. For example, if the user identifies a family history of cardiac disease (a negative indicator for cardiac health), but has a lifestyle comprised of frequent exercise and an appropriate diet (a positive indicator for cardiac health), the correlation rule will weigh these indicators against the rest of the input data to generate a functional area score for cardiac health.

The system 100 applies a correlation rule to the input data stored in the correlation database 114 and calculates the functional area score. Additionally, system 100 calculates a baseline value for each functional area. This baseline is the calculation of the highest possible positive value and the lowest possible negative value for each functional area. By comparing this baseline value to the calculated functional area score, the system is able to determine whether the positive or negative value is a priority set for the particular user. The system 100 is also capable of utilizing already calculated functional area scores as an input for calculating subsequent functional area scores. For instance, a high functional area score for a particular function may impact another functional area driving its score higher or lower as appropriate.

The functional area score database 116 stores each calculated functional area score as well as the relevant baseline value for each functional area. This data is stored every time input data is entered. The functional area scores also may be accessed during the trend analysis described above.

The report database 124 may include without limitation text and graphic content data that is entered by the administrator. The report database 124 is a repository of substantive content that is typically utilized in combination with intelligent rules to display health and wellness information to a user. The content data may be develop in a logical fashion without the administrator having to understand the inner workings of the system 100.

The content data may be utilized in the dynamic creation of output data. The content data is created by the administrator to address specific functional area patterns or scenarios identified by the system 100. The system 100 retrieves content data (as text or graphical data) for assembly to create a completely customized report for the user based upon the input data, functional area scores, and historical trends analysis.

The system 100 may include a rules engine that is capable of executing rules against input data, functional area scores, and trend data to determine the content data for the user. The rules engine may be capable of comparing the content data that has been extracted from the report database 124 to ensure that duplicate content data is not presented to the user. The rules engine is capable of ensuring that specific nutrient information, such as dosages, are listed at the level scientifically shown to address the most complex functional area. For example, if two functional areas are indicated as area of concern, both of which science indicates may be ameliorated with Vitamin C but at different dosages, the rules engine will determine and present the dosage proven to address both functional areas appropriately.

The system 100 may utilize a rules engine to analyze the functional area scores. When a more focused output data is required, multiple intelligent rules engines may be utilized to analyze input data and functional area scores. The result being thorough yet narrowed output data directed to the user.

A supplement database 128 stores supplement data on supplements the user is currently taking and their nutritional content. If the user is taking a supplement that is not found in the supplement database 128, the user has the ability to enter the label information of the supplement.

Prior to becoming available, the stored user entered label information may be validated and approved by the administrator for accuracy. The supplement database 128 also may be updated by the administrator. Additionally, direct manufacturer data feeds as well as clinic data feeds may be used to populate the supplement database 128, but as with the supplement label data entry tool 218, the supplement data may be validated by an administrator prior to becoming available.

The system 100 queries the supplement database 128 for all supplements the user indicated as currently being taken. The system 100 analyzes all the nutrients identified by the user as being consumed, matching them to the nutrients in the supplements currently being taken. A report is created for the user providing information on what supplements the user may consider continuing, what supplements the user may choose to add, and what supplements may not be a priority or may be discontinued based on the functional area scores, input data, and physician recommendations.

The system 100 utilizes a plurality of dynamic report generators that assemble the content data to be utilized in the dynamic creation of output data. The dynamic report generators dynamically build a graphically pleasing intelligible report for the user that details their individualized custom plans. The report is dynamically produced whenever the user requests it and is presented in a report viewer such as without limitation a standard internet browser. The dynamic nature of the report generation allows the user to receive the latest up to date output data every time the user requests to view their report. This is accomplished by using the stored input data as well as the stored functional scores to compile a report using the latest updated content data from the report database 124.

A user 102 may access to the system via a secure socket connection to a web server running on the system 100. A web-based graphic user interface allows the user to answer a dynamically changing questionnaire by entering input data. The input data is transformed by the system 100 to create output data. The output data is presented to the user in the form of an online report displayed by the web server over the secure socket connection.

A user 102 may access the system 100 from a doctor's clinic or office. The user 102 may connect via a secure socket connection to a web server executing on the system 100. In this embodiment, a doctor may also connect via a secure socket connection to add additional input data and/or access the user's output data to approve and/or modify the output data prior to compiling a report for the user. The report is presented to the user 102 in the form of an online report displayed by the web server over the secure socket connection.

A doctor input tool allows the physician to enter additional information into the input data. The information may include data such as laboratory data, diagnosis data, and physical evaluation data. The additional information provided by the doctor is stored in the correlation database 114 and is utilized as input data.

An output manipulator may be utilized by the user's doctor to further customize the output data for the user. The output manipulator is accessed by the doctor using a secure socket connection through a web server executing on the system 100. Using the output manipulator, the doctor can select what content data is be presented to the user. For sample, the doctor may decide not to present the user with certain diet or exercise plan because of physical constraints that were observed while evaluating the patient. The doctor is also able to add additional content to the users report via the output manipulator. For example, a prescription or other information may be added by the doctor. The information can then be added and stored in the user's report.

The report database 124 stores the modified output data in viewable and printable forms for future reference by the user or the doctor. The output data is also stored in raw data form to be used for trend analyzes and dynamic adjustments to the expert system engine and to create a customized user experience for future user interactions. The content of the clinical user's reports are stored in viewable and printable form for the user and doctor to access.

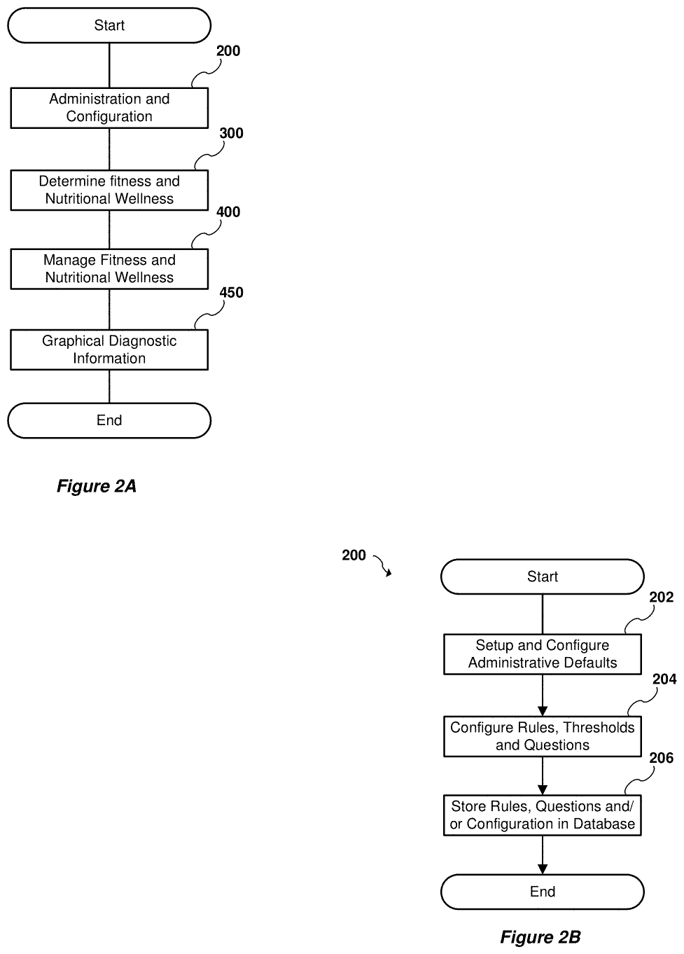

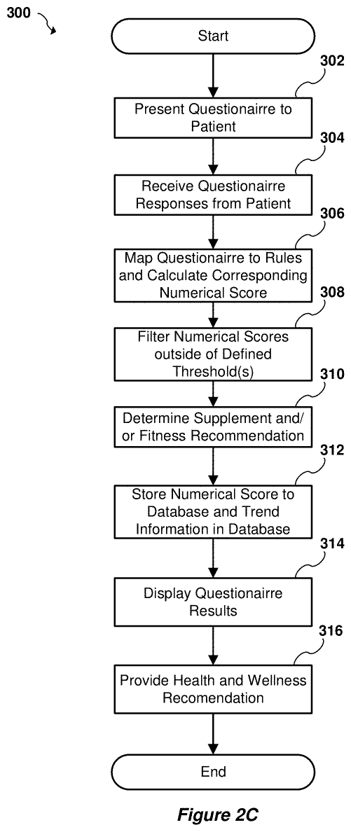

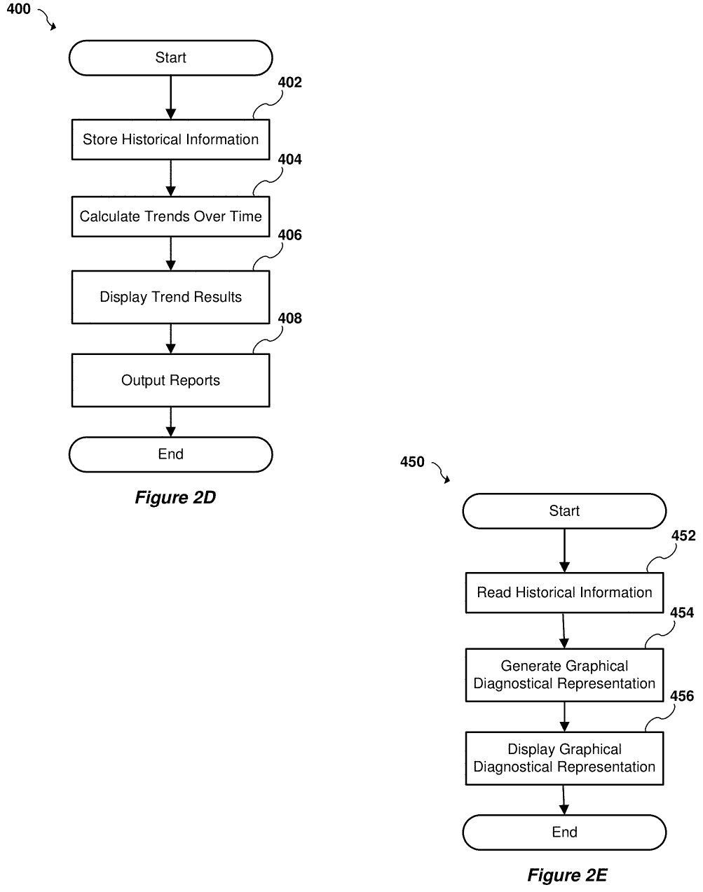

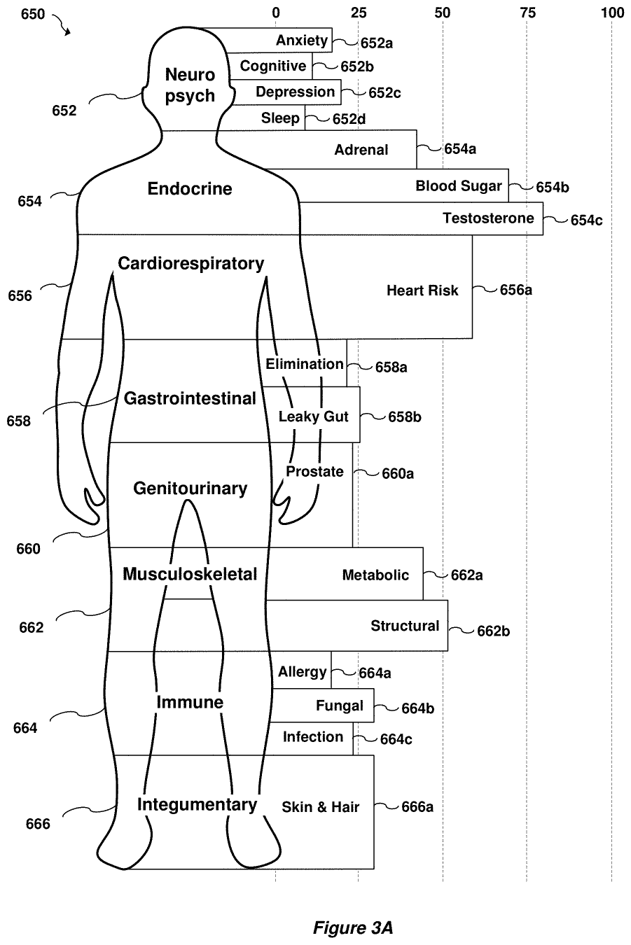

Referring to FIGS. 2A-2E, flow charts illustrating a method for analyzing and managing fitness and nutritional wellness in accordance with an embodiment of the present invention, are shown. The system 100 includes modules without limitation for administration and configuration 200, determining fitness and nutritional wellness 300, managing fitness and nutritional wellness 400, and for providing graphical diagnostic information 450. These modules may be accessed in any order.

The administration and configuration 200 module is used to setup and configure the system 100. At block 202, the system 100 receives setup and configuration information. Such setup and configuration information may include without limitation branding information, store options, managing sites, loading zip files, autoresponders, and changing passwords. The setup and configuration information also include without limitation managing clients, signup forms, supplements, report options, fitness plans, and eating plans. The rules, thresholds and questions are configured at block 204. At block 206, the rules, questions and configuration are stored in the database 110.

The fitness and nutritional wellness determination 300 module is used to determine the fitness and wellness of the patient. At block 302, the system 100 presents a questionnaire to a patient. The responses to the questionnaire is received at block 304. At block 306, the responses to the questionnaire are mapped to the appropriate rules and a numerical score is calculated. The numerical scores outside of defined thresholds are filtered at block 308. At block 310, based on the numerical scores, a fitness plan, eating plan and/or supplement(s) are determined by the system 100. The numerical score and trend information is stored in the database 110 at block 312. At block 314, the questionnaire results are displayed. The results may be displayed as a report, a graphical chart, numerical scores, and the like. The user is provided a health and wellness recommendation to the user 102.

The managing fitness and nutritional wellness 400 module is used to manage and maintain a patient's health and wellness. At block 402, historical information is stored in the database 110. The trends are calculated and displayed at blocks 404 and 406. At block 408, the results are displayed to the user. The results may be displayed as a report, a graphical chart, numerical scores, and the like.

The graphical diagnostic information 450 module is used to generate a graphical representation of a patient's health and wellness. At block 452, historical information is read from the database 110. At block 454, a graphical diagnostical representation is generated as described below. The graphical diagnostical representation is displayed to the user at block 456.

### Graphical Diagnostic Representation

According to the present invention, the graphical diagnostic information 450 graphically representing a patient's health and wellness is based on the exemplary functional systems as described in Table 3 below.

TABLE 3
FUNCTIONAL SYSTEMS

| Functional System | Description |
|---|---|
| Cardiorespiratory | The cardiorespiratory system provides |
| | oxygenated blood and |
| | nutrients to tissues and removes |
| | waste products from this process. |
| Endocrine | The endocrine system releases |
| | hormones (chemical messengers) that |
| | communicate with every cell in the |
| | body to regulate metabolism, |
| | sexual function and thought. |
| Gastrointestinal | The gastrointestinal (GI) system |
| | processes food through the phases of |
| | digestion, absorption and elimination. |
| | The GI system also produces |
| | neurotransmitters and assists |
| | in detoxification. |
| Genitourinary | The genitourinary system regulates |
| | reproduction, the pH and volume |
| | of blood and elimination of waste. |
| Immune | The immune system defends against |
| | infection, balances inflammation |
| | for healing and harmonizes the |
| | environment inside the body with the |
| | outside world. |
| Integumentary | The integumentary system is the |
| | skin, hair and nails that form a |
| | barrier to the outside world and |
| | regulate temperature. |
| Musculoskeletal | The Musculoskeletal system |
| | consists of muscle, bone, marrow, |
| | cartilage, ligament and tendon. |
| | The Musculoskeletal system supports |
| | and protects internal organs, balances |
| | mineral content and generates |
| | heat through muscle contraction. |
| | Mesenchymal stem cells in bone |
| | marrow form red and white blood |
| | cells, platelets and progenitor cells |
| | for all musculoskeletal components. |
| Neuropsychiatric | The Neuropsychiatric system |
| | communicates, coordinates and |
| | responds to sensation. This system |
| | generates mood and cognition |
| | (thought). |

Each of the above exemplary function systems have one or more exemplary health alerts shown in the exemplary Tables 4-11 below. These health alerts are typically brought to a doctor's attention and belong to specific functional systems.

TABLE 4
CARDIORESPIRATORY HEALTH ALERTS

| Health Alert | Description |
|---|---|
| Heart Risk | Heart risk can be lowered through |
| | taking great care of this critical |
| | muscle. Eating foods that balance |
| | blood sugar reduces inflammation |
| | and preserves arterial function. |
| | Sustained aerobic exercise at a safe |
| | level for your current state of health |
| | builds the strength of your heart |
| | muscle and stabilizes the entire |
| | blood vessel system. Add nutrients |
| | that promote healthy heart muscle |
| | including Coenzyme Q10, |
| | magnesium, fish oil with Omega 3 fatty |
| | acids, selenium and vitamin E. |

TABLE 5
ENDOCRINE HEALTH ALERTS

| Health Alert | Description |
|---|---|
| Adrenal | Adrenal Imbalance can be improved |
| | by reducing physical and |
| | emotional stress. Eat a little protein |
| | with every meal and journal your |
| | mood and energy level. Your diet |
| | should always be adjusted to keep |
| | you calm and focused. Gentle regular |
| | physical activity will also tone |
| | the adrenal gland and even out elevated |
| | adrenaline levels. |
| Blood Sugar | Blood sugar and high Insulin levels |
| | can often be reduced if you |
| | increase your physical activity |
| | and reduce the amount of |
| | carbohydrates in your diet. A mix of |
| | aerobic and resistance (weight) |
| | training will give you the best |
| | result for insulin reduction. |
| | Supplements that help sugar metabolism |
| | can reduce insulin levels as well. |
| | Vanadium, chromium and L-glutamine |
| | have all proven to be |
| | useful in balancing cravings and insulin |
| | levels. High blood sugar and |
| | insulin levels are very commonly |
| | related to improper food selection |
| | and can be rapidly improved with |
| | intentional eating strategics. |
| Estrogen | Low Estrogen Activity is often |
| | associated with menopause but may |
| | be caused by any stressor at nearly |
| | any age. The ovaries are the |
| | primary producer of estrogens after |
| | puberty but the adrenal gland and |
| | fatty tissues also contribute all |
| | throughout the lifespan. Supporting |
| | adrenal function through stress |
| | reduction and other means can be a |
| | safe and effective way to reverse |
| | low estrogen status. Over-the- |
| | counter herbal treatments that |
| | contain Black Cohosh and other |
| | compounds can be very helpful as well. |
| Progesterone | Low progesterone activity is often |
| | associated with stress or excess |
| | exposure to estrogenic compounds |
| | in the diet. Reducing refined |
| | carbohydrates and sugar in your |
| | diet is a good first step. Increasing |
| | aerobic exercise and relaxation |
| | techniques, including deep breathing |
| | can help. Symptoms of PMS are |
| | often associated with low |
| | progesterone levels and progesterone |
| | replacement is often more |
| | effective than drug therapy in |
| | these situations. |
| Testosterone | Testosterone Imbalance can develop |
| (Female) | at any age after puberty and |
| | frequently in early menopause. |
| | Weight training and resistance |
| | exercise in conjunction with weight |
| | loss can improve testosterone |
| | levels. Avoiding alcohol and processed |
| | foods in combination with |
| | stress reduction can also increase |
| | testosterone levels. |
| Testosterone | Low testosterone (male menopause/ |
| (Male) | andropause) can develop at any |
| | age after puberty. Weight training |
| | and resistance exercise in |
| | conjunction with weight loss can |
| | improve testosterone levels. |
| | Avoiding alcohol and processed |
| | foods in combination with stress |
| | reduction can also increase |
| | testosterone activity. Zinc, DHEA, |
| | Omega-3 oils, selenium, vitamin |
| | D, E and lycopene can all have an |
| | effect on testosterone. |
| Thyroid | Thyroid activity is often low from |
| | problems like Grave's disease but |
| | more often than not, thyroid function |
| | is inadequate to meet the needs |
| | of a stressful environment simply |
| | because of external factors. Body |
| | temperature testing and complete |
| | blood testing including Free T3, |
| | Free T4 and TSH are usually enough |
| | to be certain that the thyroid |
| | gland is working at its optimal |
| | level. Though TSH levels may be in |
| | the upper normal range (2-4.5) the |
| | thyroid may not be providing |
| | optimal support. |

TABLE 6
GASTROINTESTINAL HEALTH ALERTS

| Health Alert | Description |
|---|---|
| Digestive Enzymes | Low digestive enzyme activity may |
| | be at the root of your health |
| | challenges. As much as possible, |
| | eat fruits and vegetables raw or |
| | very lightly cooked so their natural |
| | enzymes make them easy to |
| | digest. Take digestive enzymes right |
| | in the middle of meals and |
| | especially with all cooked food. |
| | Stress reduction helps the digestive |
| | system work more effectively-so |
| | breathe deeply. Avoid late night |
| | snacking when enzyme levels are |
| | at their lowest point. |
| Elimination (slow) | Slow elimination (often with |
| | constipation) makes it difficult to |
| | remove toxins and waste. Healthy |
| | people can improve their bowel |
| | function by increasing fluids and |
| | fruits and vegetables that are high in |
| | fiber. Vigorous physical activity of |
| | any kind helps the bowel move |
| | more effectively. |
| Leaky Gut | Leaky Gut improves when foods |
| | that cause allergic responses and |
| | irritation are removed from your diet. |
| | Wheat, dairy, sugar and soy |
| | commonly cause problems. Avoid |
| | fast foods that are loaded with |
| | pesticides, hormones and antibiotics. |
| | Eat organic when possible to |
| | enhance nutrition and reduce pesticides. |
| | Get up and move to get your |
| | gastrointestinal tract active and reduce |
| | stress. Supplementing with |
| | good bacteria (probiotics) and taking |
| | L-glutamine can help to heal |
| | the lining of the intestine. |

TABLE 7
GENITOURINARY HEALTH ALERTS

| Health Alert | Description |
|---|---|
| Prostate | Prostate enlargement makes urination |
| | difficult to start, continue and |
| | stop. Pain is usually not a problem |
| | though checking for prostate |
| | cancer with a simple PSA blood test |
| | and physical exam is a good |
| | starting point. |

TABLE 8
IMMUNE HEALTH ALERTS

| Health Alert | Description |
|---|---|
| Allergy | Allergy problems are often caused by |
| | foods. that irritate the immune |
| | system. Milk, wheat, corn and soy are |
| | commonly discovered as a |
| | root cause of allergies during blood |
| | testing. Dust and environmental |
| | allergens like pollen and molds irritate |
| | the immune system through |
| | the breathing passages. Stress reduction |
| | improves the output of the |
| | body's own steroids and can create |
| | large and lasting benefits. |
| | Nutrients like quercetin, stinging |
| | nettle extract and vitamin C |
| | frequently improve the symptoms of allergy. |
| Fungal | Excessive fungal load can be addressed |
| | by eating less refined |
| | carbohydrate (especially sugar). This |
| | may be the most important step |
| | you can take to control the growth |
| | of fungus. Sweet fruits and root |
| | vegetables may also contribute to the |
| | growth of fungus though they |
| | are not nearly as damaging as sugar. |
| | Stress reduction with deep |
| | breathing and gentle exercise enhances |
| | immune response and creates |
| | a hostile environment for fungus. |
| | Taking plant-based nutrients like |
| | olive leaf extract, grapefruit seed |
| | extract and uva ursi can help keep |
| | fungus in check. Medications like |
| | nystatin and Diflucan may be |
| | helpful when natural remedies |
| | aren't effective. |
| Infection | Infection with bacteria, virus or |
| | parasite can be chronic (lasting |
| | years) or acute (days). Infection |
| | can be controlled by eating less |
| | sugar and processed foods. This |
| | denies food to bad bacteria in the |
| | stomach and intestines. Stress |
| | reduction supports a healthy immune |
| | system that can repel viral, parasitic |
| | and bacterial invaders before |
| | they become deeply embedded |
| | in the tissues of every system. |
| | Aerobic activity and deep breathing |
| | improve circulation to the tissues |
| | and enhance drainage of infectious |
| | waste. Supplementation with |
| | vitamin C, grapefruit seed extract, |
| | garlic and probiotics make it |
| | difficult for bacteria to set up shop |
| | in the intestines. |

TABLE 9
INTEGUMENTARY HEALTH ALERTS

| Health Alert | Description |
|---|---|
| Skin and Hair | Skin and hair changes often suggest |
| | underlying stress from aging and |
| | infection. Though skin changes are |
| | often treated as a local problem |
| | they are often deep-seated immune |
| | and digestive issues. |

TABLE 10
MUSCULOSKELETAL HEALTH ALERTS

| Health Alert | Description |
|---|---|
| Metabolic | Metabolic imbalances in magnesium |
| | and calcium can cause bone loss |
| | and pain. Inflammation in joints |
| | and other connective tissues can |
| | create myofascial pain syndromes, |
| | rheumatoid disease and other |
| | auto-immune changes. Inflammation |
| | turns on healing through |
| | immune activation and stem |
| | cell signaling. |
| Structural | Structural (musculoskeletal and c |
| | onnective tissue) problems reduce |
| | function through pain, poor flexibility |
| | and effects on the internal |
| | organs and systems. Stretching in a |
| | pain-free range of motion is a |
| | key to maximizing structural health. |
| | Professional “body-workers” in |
| | the fields of Osteopathic, Chiropractic |
| | and Physical Therapy can all |
| | provide great insight as well as |
| | treatment. Supplements to enhance |
| | structural healing include digestive |
| | enzymes, bromelain and Vitamin |
| | C. Quality proteins build and repair |
| | muscle along with nutrients that |
| | support connective tissues like |
| | ligaments and joints. |

TABLE 11
NEUROPSYCHIATRIC HEALTH ALERTS

| Health Alert | Description |
|---|---|
| Anxiety | Anxiety is a sense of worry, fear, |
| | doubt or even dread. Deep |
| | breathing is the best technique |
| | to immediately reduce anxiety. As |
| | deep breathing stretches nerve |
| | bundles and improves brain |
| | circulation the body reflexively |
| | relaxes. Anxiety can also be reduced |
| | by eliminating caffeine and other |
| | stimulants from the diet. |
| Cognitive | Cognitive Problems range from |
| | misplacing the car keys because of |
| | excessive stress to the frightening |
| | possibility of Alzheimer's disease. |
| | Most mild cognitive problems |
| | can be quickly addressed through |
| | targeted nutrition including |
| | brain-specific nutrients that can restore |
| | normal thought and recall. With |
| | foods, exercise and brain-building |
| | activities the fear of life-changing |
| | thought disorders can be banished. |
| Depression | Depression is usually caused by a |
| | combination of brain chemistry, |
| | emotion and environment. Increasing |
| | physical activity and removing |
| | toxic foods from the diet can make |
| | a dramatic change in mood. |
| | Specific nutrients can support brain |
| | chemistry and boost levels of |
| | serotonin and other crucial |
| | neurotransmitters. Depressed mood isn't |
| | always as simple as feeling “blue” |
| | and may impact every function in |
| | the body, especially the immune system. |
| Sleep | Sleep allows deep healing to occur in |
| | organ systems at specific times |
| | of the sleep cycle. |

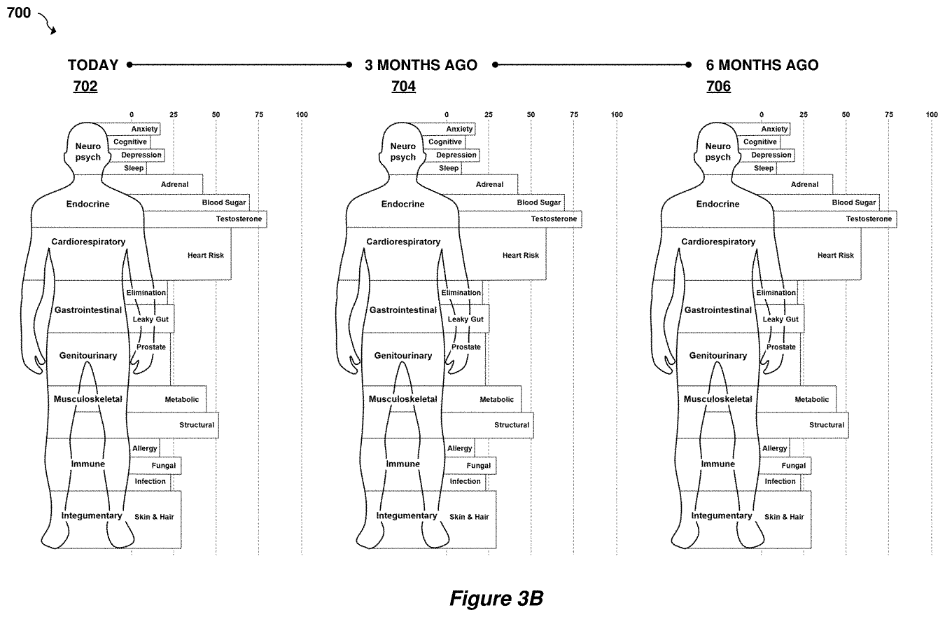

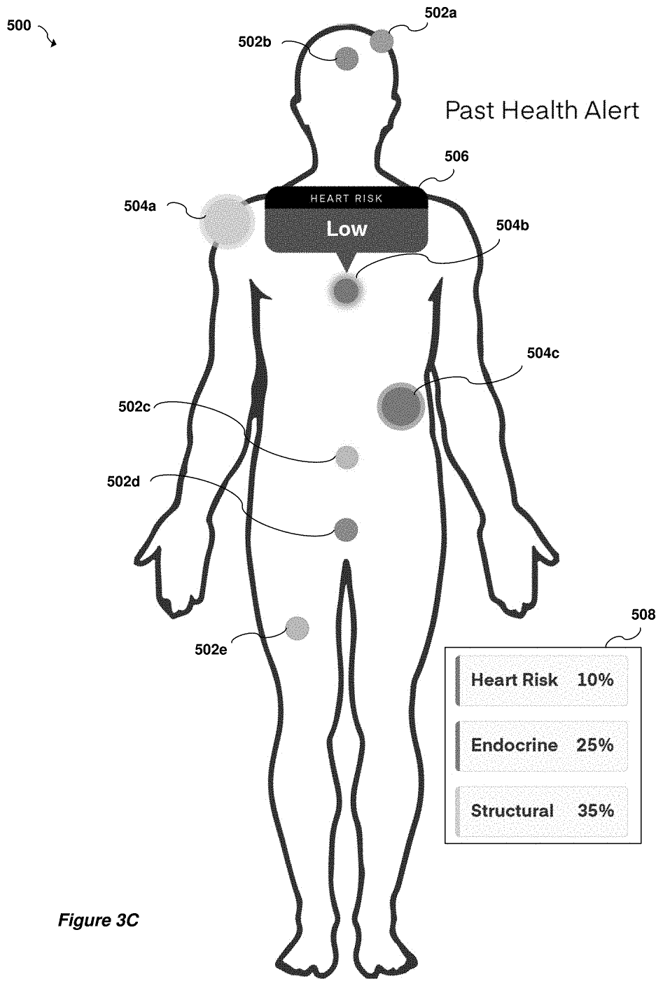

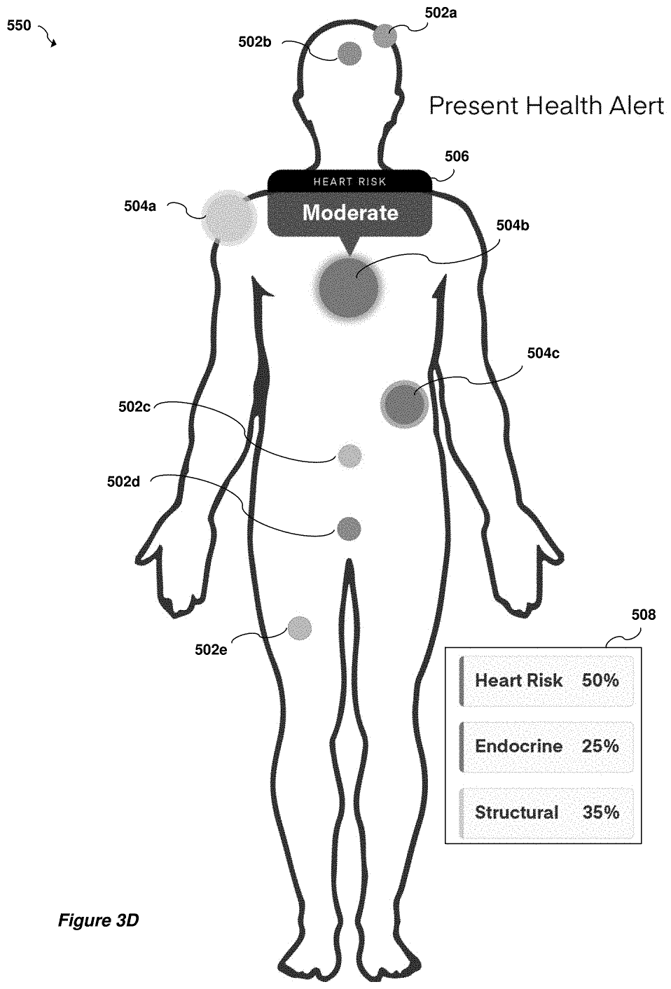

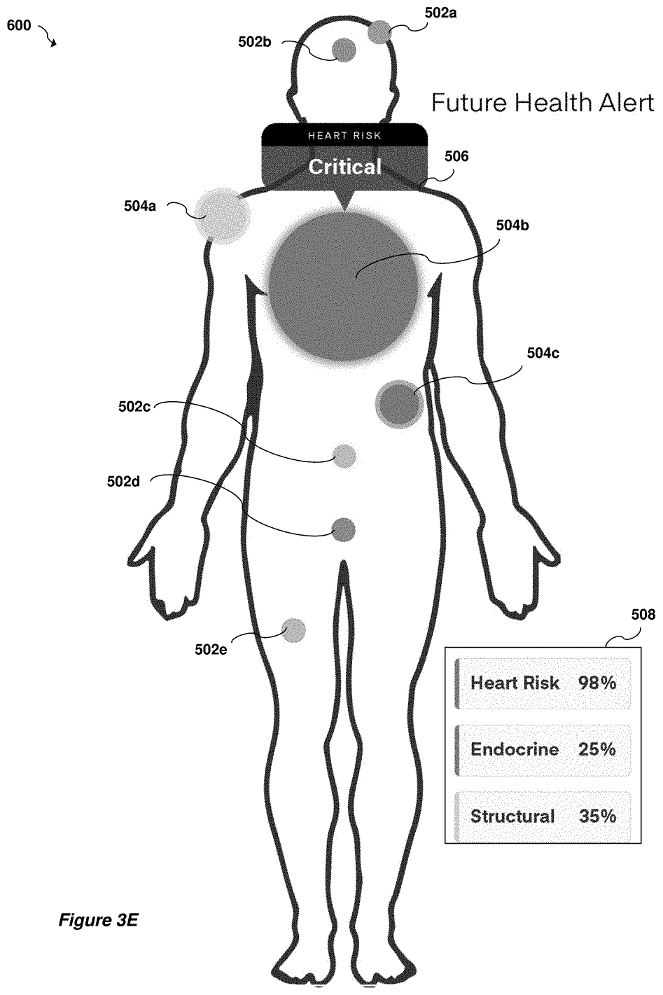

Referring to FIGS. 3A-3B, an exemplary user interface for graphically presenting fitness and nutritional wellness information in accordance with an embodiment of the present invention. According to this embodiment, the present invention utilizes a composite score resulting from symptom interview in combination with objective data which may be received from a HL7 bridge. However, it is understood that objective data may be received from other sources within the scope of the present invention. The resulting data is translated into a diagnostic image generator for the purpose of creating a graphical diagnostical representation of the many layers of complex symptomatic and objective data. The many layers of complex symptomatic and objective data can be analyzed graphically by the human eye. According to one embodiment, the present invention maintains historical information over a prescribed period of time for a patient. This historical information is used by the present invention to generate images for evaluation over a prescribed time period.

The present invention applies an interpretive algorithm to all classes of data to build an image that summarizes all of the data and diagnostic probabilities. A diagnostic image is presented to the human eye. The subtle inferences and nuances of the human mind are employed to create the final health and wellness conclusions with respect to several key body systems.

As shown in FIG. 3A, a graphical diagnostical representation of the patient's functional systems may include, without limitation, the neuro psych 652, endocrine 654, cardiorespiratory 656, gastrointestinal 658, genitourinary 660, musculoskeletal 662, immune 664 and integumentary 666 functional systems. The neuro psych 652 functional system representation includes, without limitation, representations for anxiety 652a, cognitive 652b, depression 652c and sleep 652d health alerts. The endocrine 654 functional system representation includes, without limitation, representations for adrenal 654a, blood sugar 654b and testosterone 654c. The cardiorespiratory 656 functional system representation includes, without limitation, a representation for a heart risk 656a health alert. The gastrointestinal 658 functional system representation includes, without limitation, representations for elimination 658a and leaky gut 658b health alerts. The genitourinary 660 functional system representation includes, without limitation, a representation for a prostate 660a health alert. The musculoskeletal 662 functional system representation includes, without limitation, representations for metabolic 662a and structural 662b health alerts. The immune 664 functional system representation includes, without limitation, representations for allergy 664a, fungal 664b and structural 664c health alerts. The integumentary 666 functional system representation includes, without limitation, a representation for a skin and hair 666a health alert.

According to a non-limiting embodiment, Table 12 below provides possible color representations of the functional body systems. However, it is understood that other color representations are possible within the scope of the present invention.

According to an alternative non-limiting embodiment, each of the functional systems (652-666) may be further represented by different colors representing a patient's health condition in each of these areas. For instance, different colors may be used to represent different neuro psych 652 functional system conditions, such as, without limitation, red for an unhealthy condition, yellow for a warning condition, and green for a healthy condition. However, is understood and any number of colors may be utilized to represent differing health conditions without departing from the scope of the present invention.

According to a non-limiting embodiment, each of the health alerts (652a-652c, 654a-654c, 656a, 658a-658b, 660a, 662a-66b, 664-664c and 666a) are represented by a bar in a bar chart visually indicating the degree of health alert in each of those areas and numerically ranging from 0 to 100, where 0 represents a healthy condition and 100 represents a critical condition. For instance, as shown in FIG. 3A, the testosterone 656c health alert represents a fairly unhealthy condition with a longer bar representation reflecting a numerical score above 75, whereas, the sleep 652d health alert represents a fairly healthy condition with a shorter bar representation reflecting a numerical score below 25. Each of the health alerts (652a-652c, 654a-654c, 656a, 658a-658b, 660a, 662a-66b, 664-664c and 666a) may be further represented by different colors representing a patient's health condition in each of these areas. For instance, different colors may be used to represent different testosterone 654c health alert conditions, such as, without limitation, red for an unhealthy condition, yellow for a warning condition, and green for a healthy condition. Alternatively, different colors may be used to represent other information useful in diagnosing the patient's health condition. Thereby, a patient's overall health condition may be determined at a glance. Further, health conditions which must be immediately addressed are clearly indicated at a glance.

The present invention generates a graphical health and wellness representation using color, size and placement to enhance understanding of the patient's health and wellness status over a specified time period, including without limitation the past through the present and through the future as prediction of future events. For instance, a patient's health condition as of today 702, three months ago 704, and six month ago 706, is shown in FIG. 3B. Thereby, a visual comparison of the changes over time with respect a patient's health condition for functional systems (652-666) and health alerts (652a-652c, 654a-654c, 656a, 658a-658b, 660a, 662a-66b, 664-664c and 666a) can be ascertained at glance. This is especially helpful in determining if the patent's health condition is improving or deteriorating over time.

Referring to FIGS. 3C-3E, an exemplary user interface for graphically presenting fitness and nutritional wellness information in accordance with an alternative embodiment of the present invention. According to this embodiment, the present invention utilizes a composite score resulting from symptom interview in combination with objective data which may be received from a HL7 bridge. However, it is understood that objective data may be received from other sources within the scope of the present invention. The resulting data is translated into a diagnostic image generator for the purpose of creating a graphical diagnostical representation of the many layers of complex symptomatic and objective data. The many layers of complex symptomatic and objective data can be analyzed graphically by the human eye. According to one embodiment, the present invention maintains historical information over a prescribed period of time for a patient. This historical information is used by the present invention to generate images for evaluation over a prescribed time period. The present invention applies an interpretive algorithm to all classes of data to build an image that summarizes all of the data and diagnostic probabilities. This diagnostic image is presented to the human eye similarly to a bar code or UPC symbol would be to an electronic “reader.” The subtle inferences and nuances of the human mind are employed to create the final health and wellness conclusions. The graphical diagnostical representation may be presented without limitation as a past health alert 500, a present health alert 550, and future health alert 600. Each of these graphical diagnostical representations includes health indicators 502a-502e and 504a-504c. It may also include one or more alerts 506. Health indicators 502a-502e and 504a-504c represent body systems evaluated over time. The present invention generates a graphical health and wellness representation using color, size and placement to enhance understanding of the patient's health and wellness status over a specified time period, including without limitation the past through the present and through the future as prediction of future events.

TABLE 12
EXEMPLARY COLOR REPRESENTATION

| Function Body System | Color Representation |
|---|---|
| Cardiorespiratory | Red |
| Endocrine | Blue |
| Gastrointestinal | Tan |
| Genitourinary | Magenta |
| Immune | Green |
| Integumentary | Gold |
| Musculoskeletal | Yellow |
| Neuropsychiatric | Purple |

Patient problems or concerns within a system are predicted through symptom scoring and laboratory confirmation. Health indicators 502a-502e and 504a-504c are shown as circles and vary in size depending on probability of a problem. For instance, the size of the circle increases in size with increased likelihood and decreases in size with diminishing probabilities. Predictive scoring is extended into the future based on calculations of trajectories from previous scores and historical trending. This graphic representation is a man-machine interface meant to accelerate evaluation of extensive amounts of health data. The system's conclusions are presented to the patient and practitioner for final conclusions. The graphical representation may also include patient status information 506 and 508.

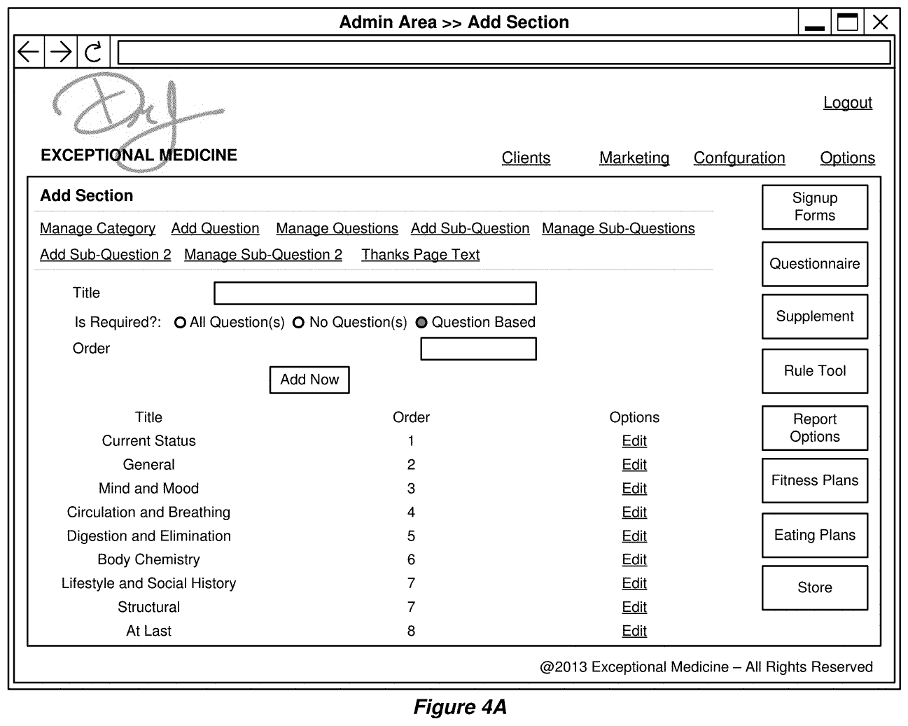

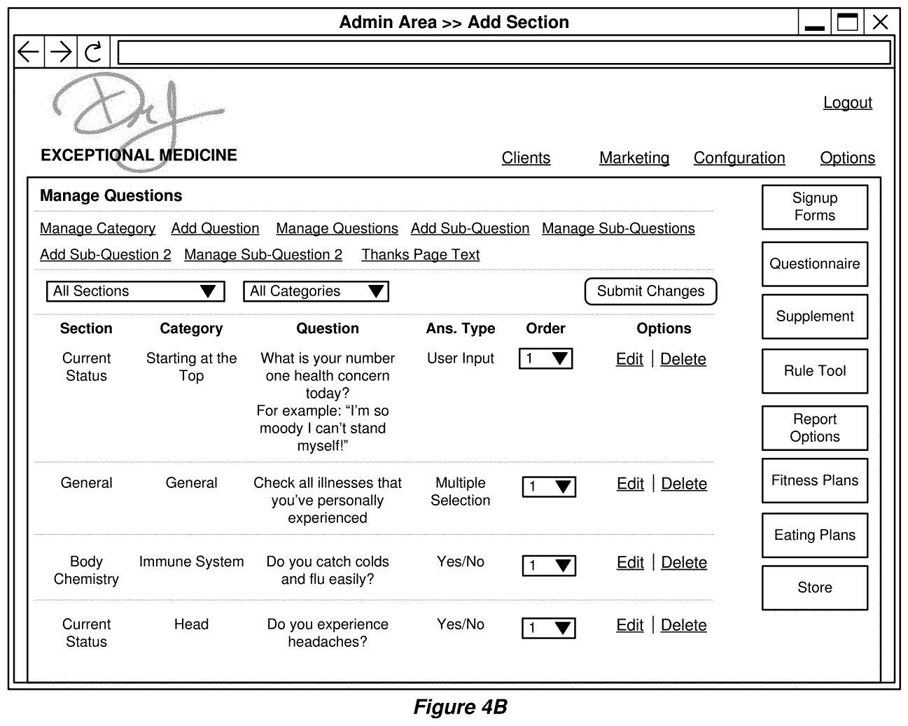

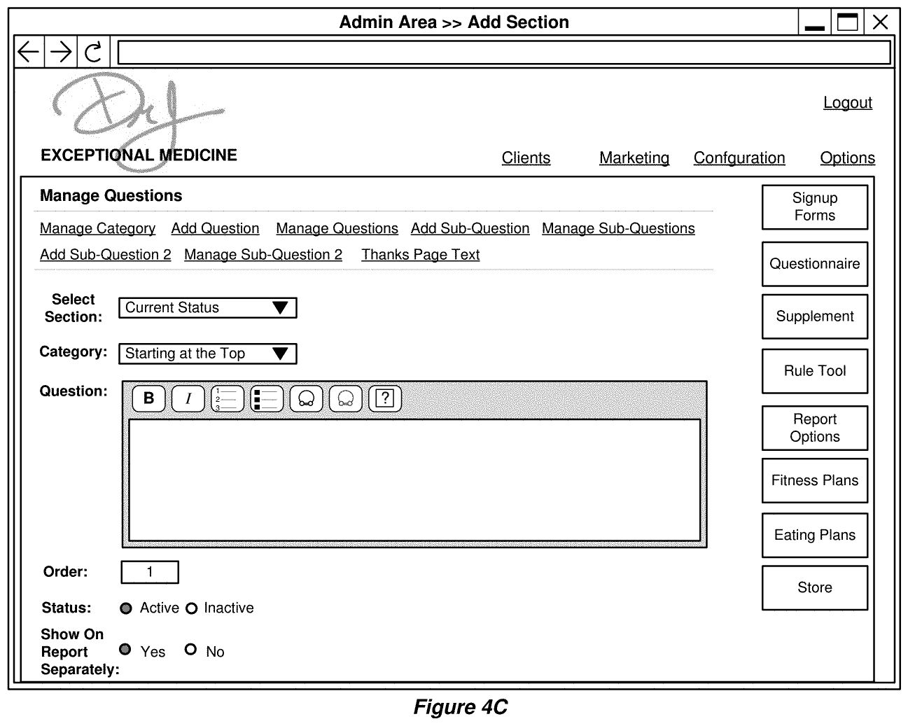

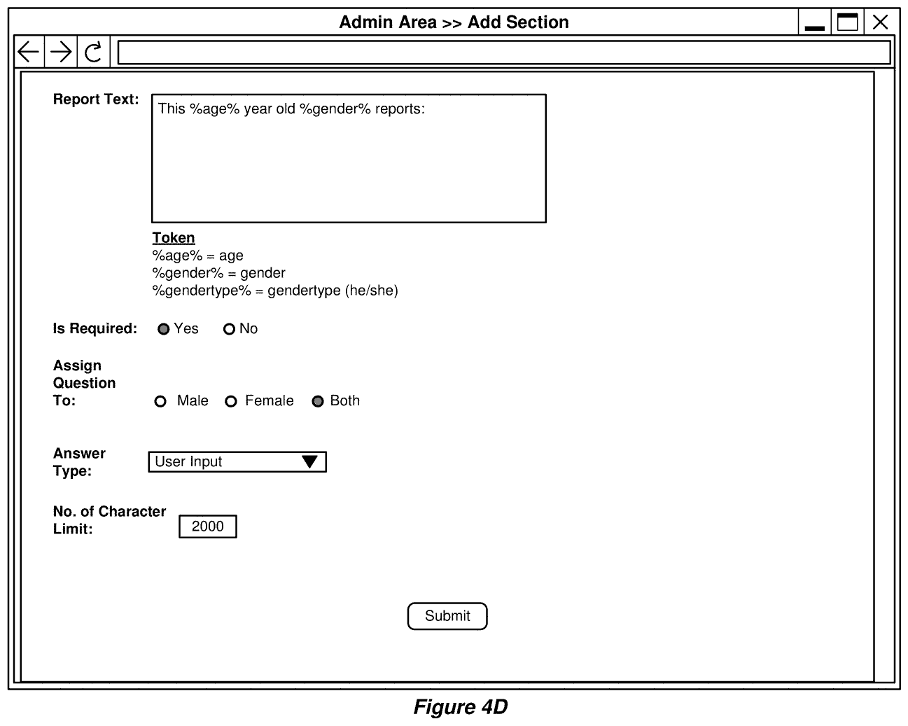

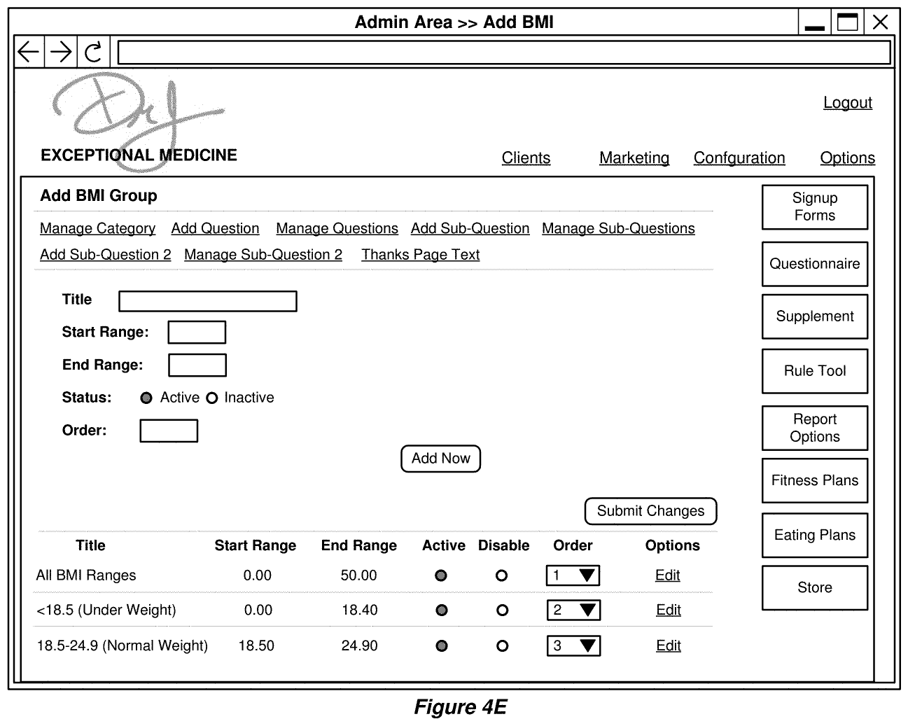

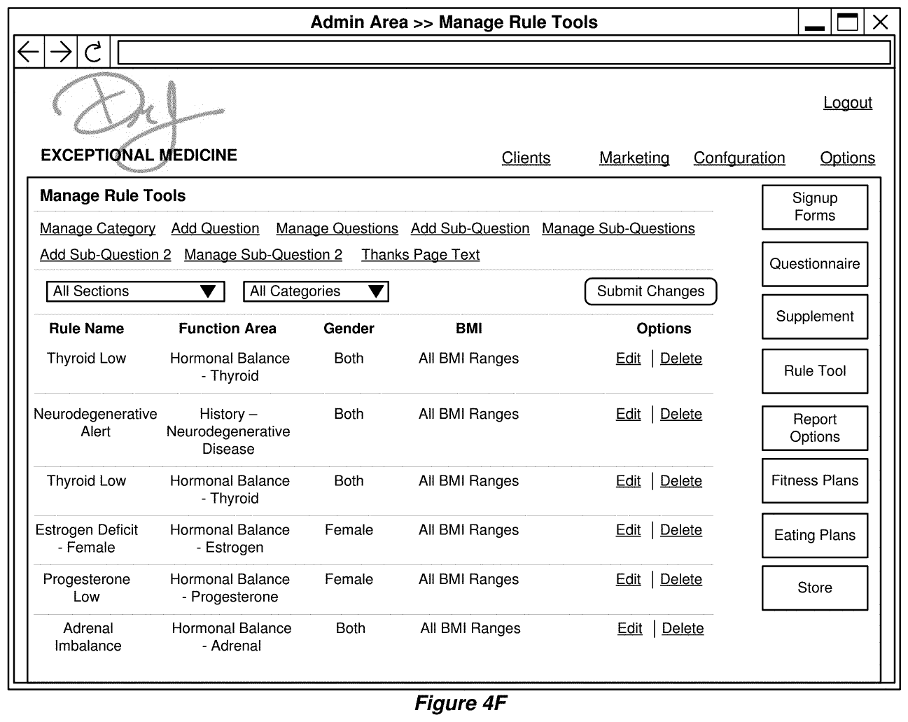

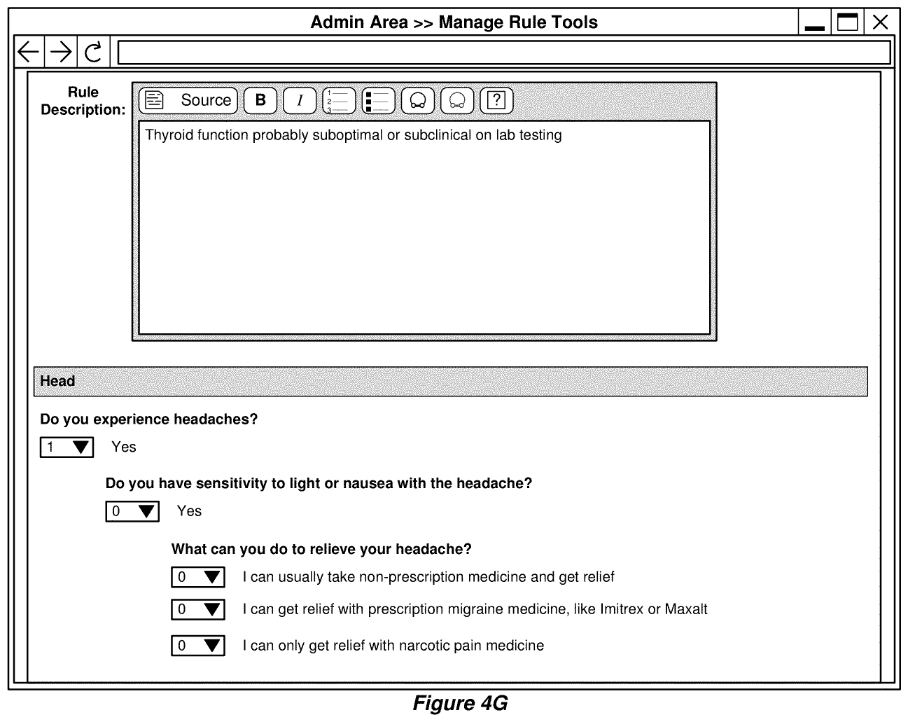

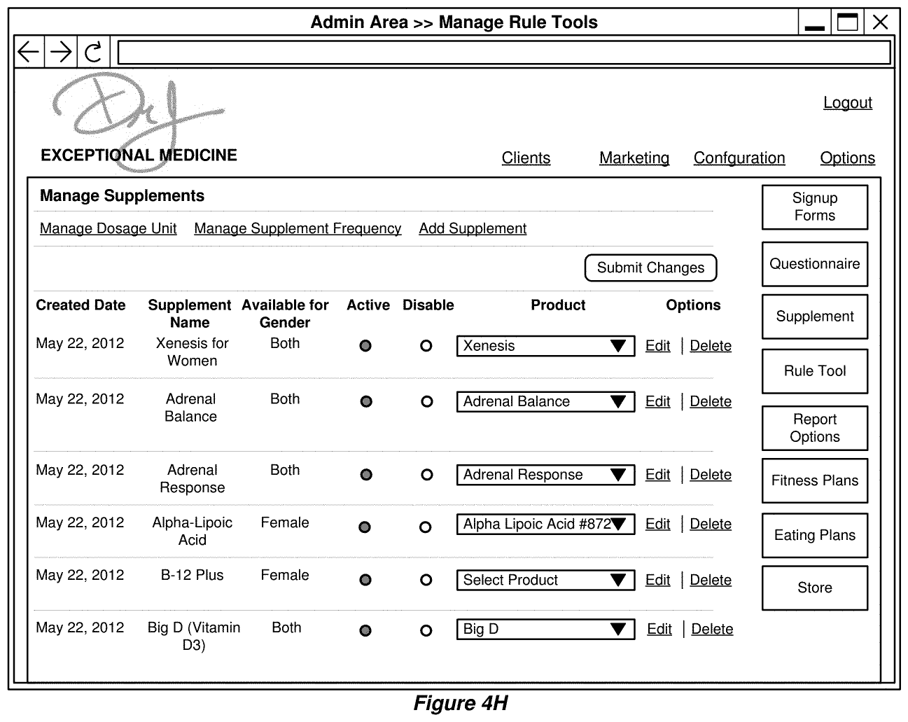

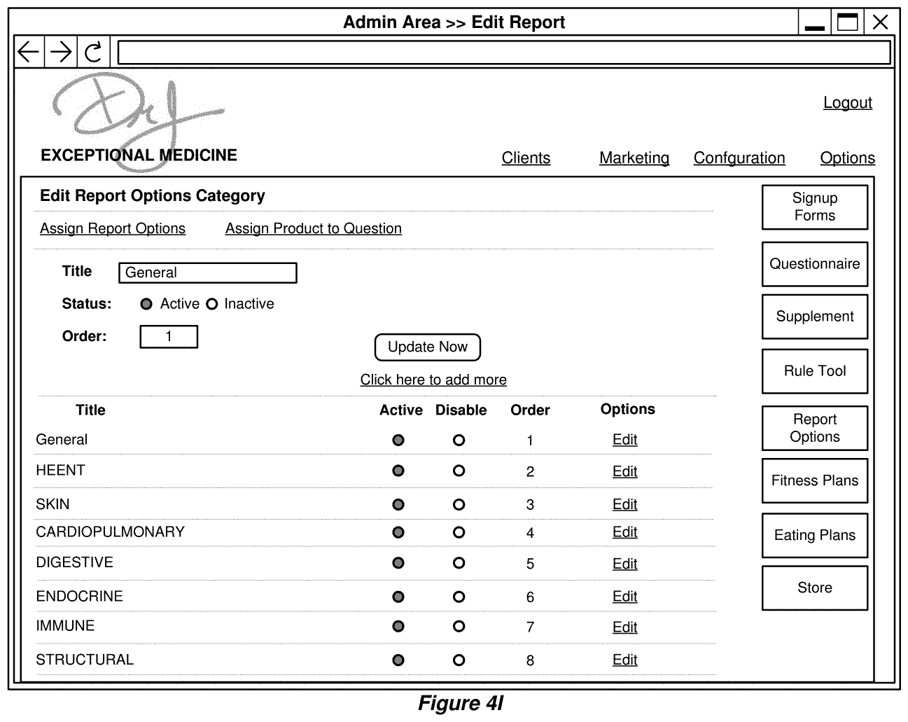

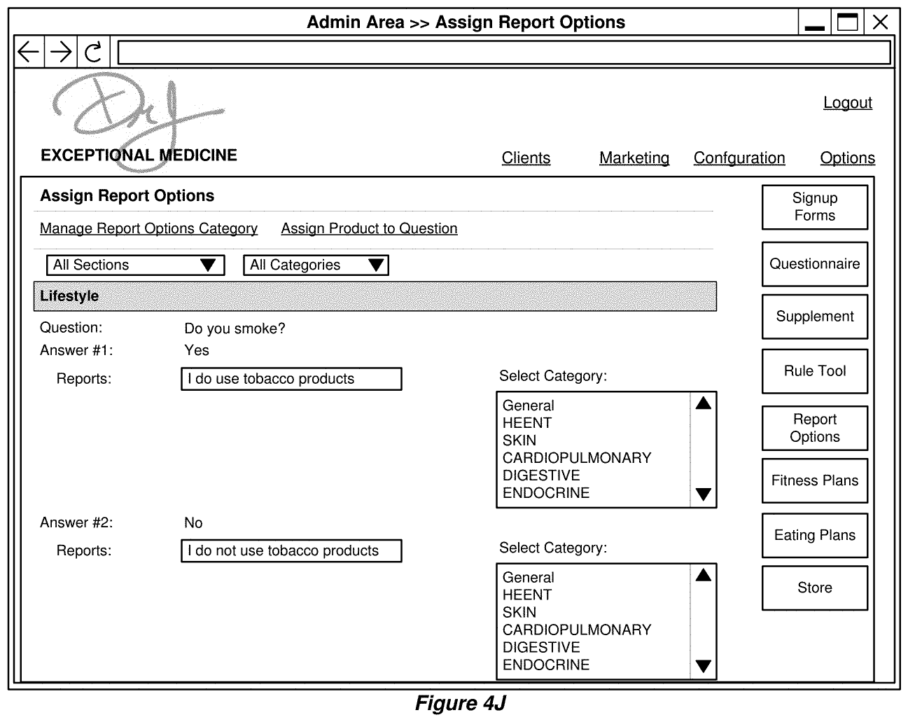

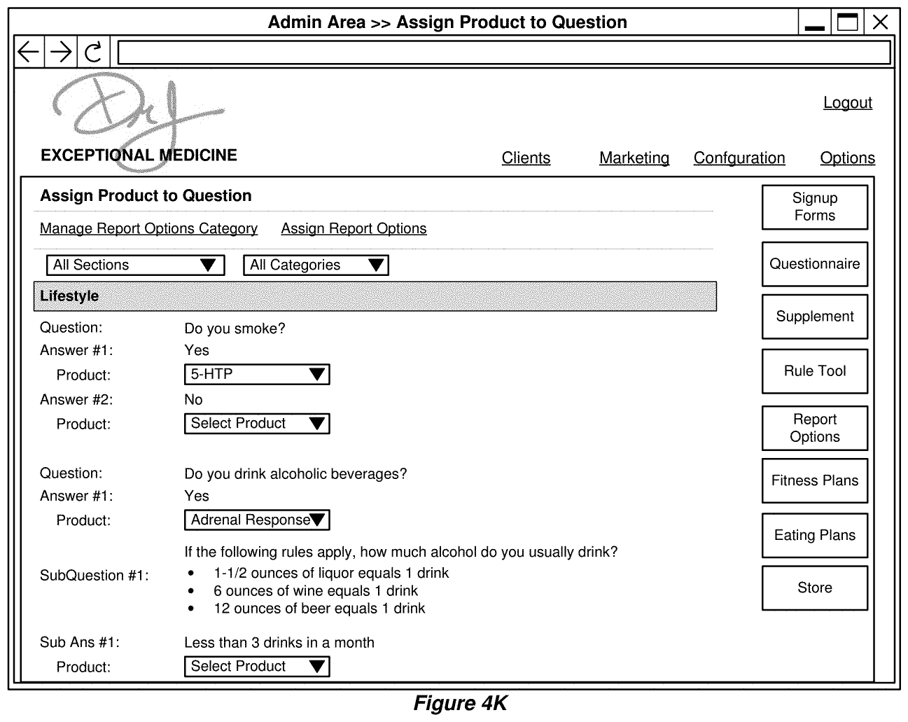

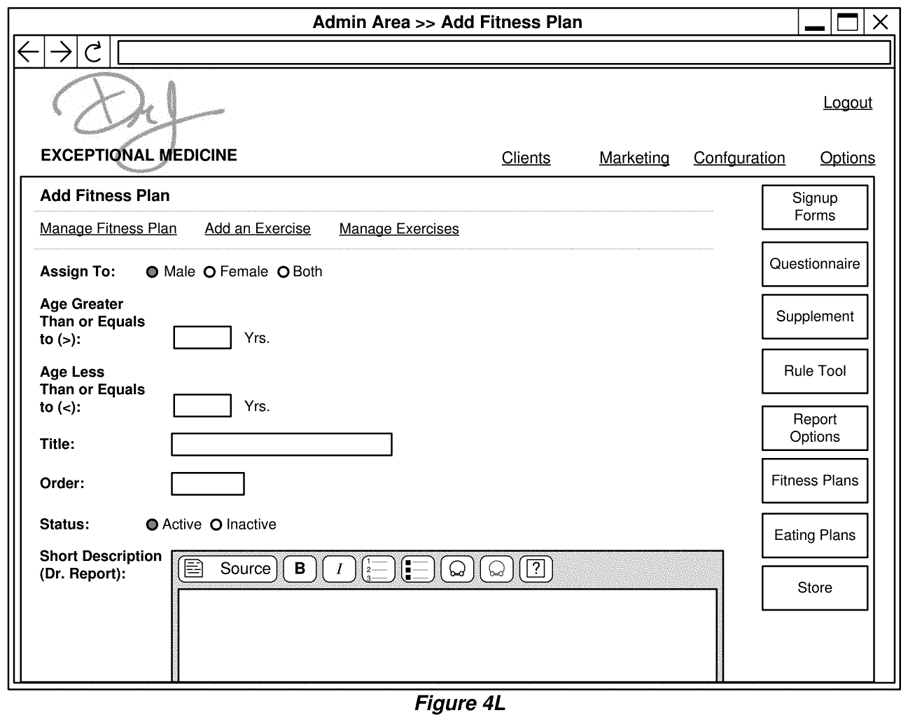

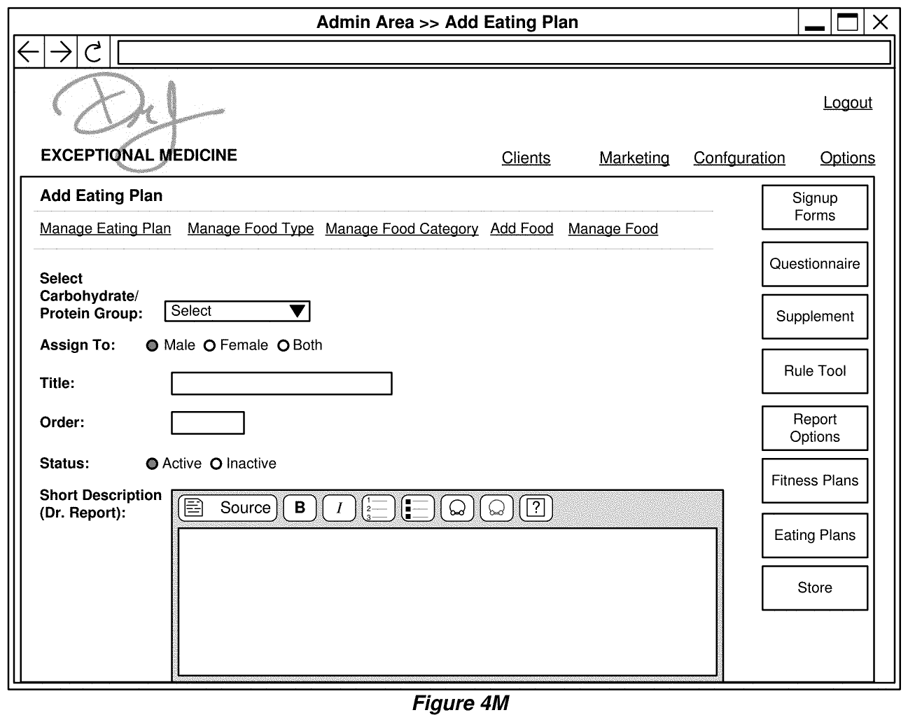

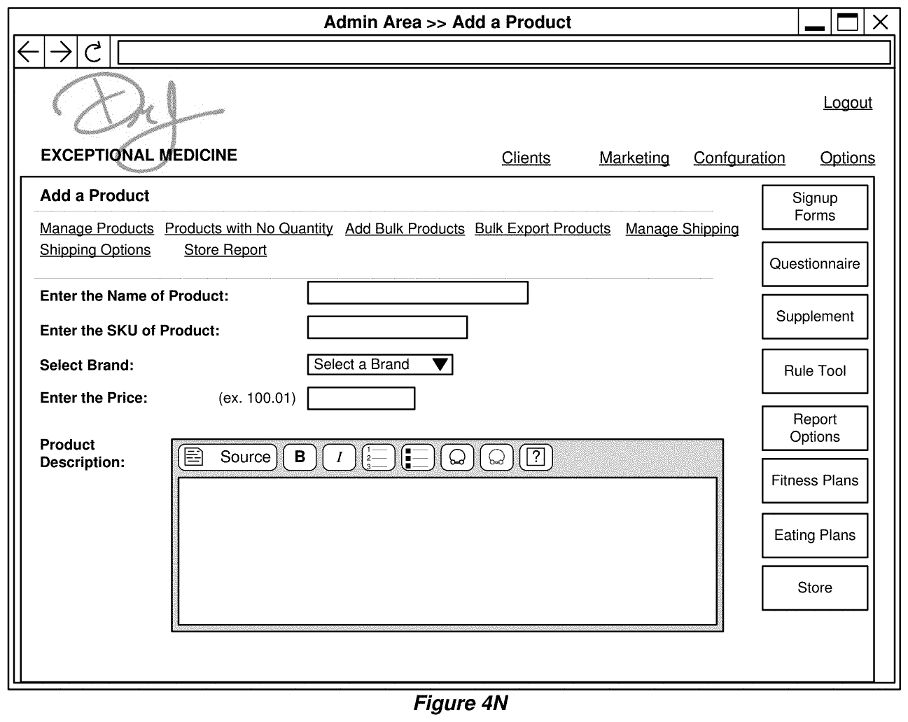

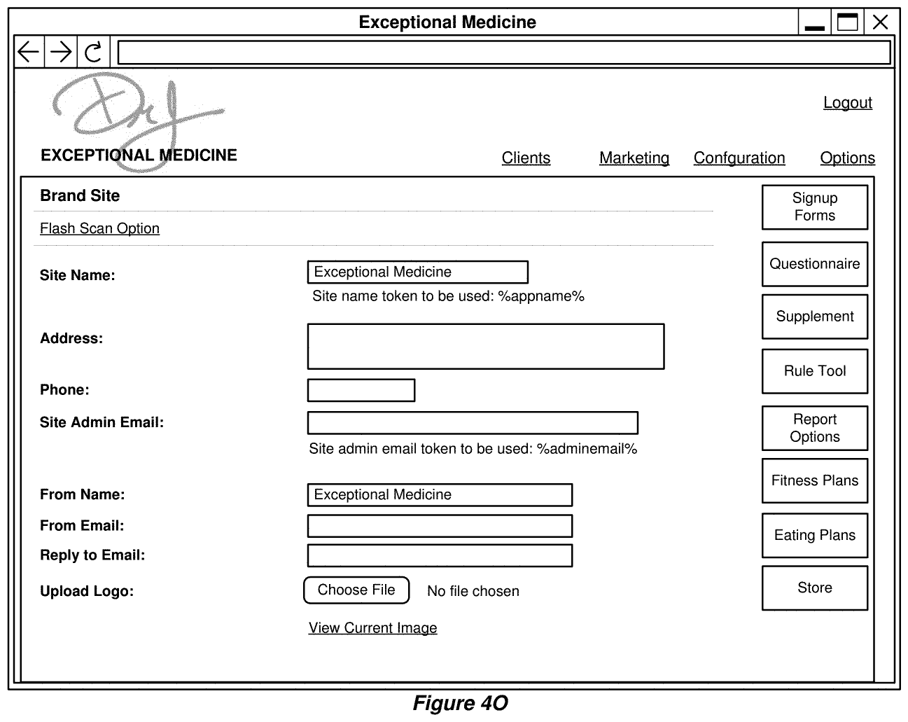

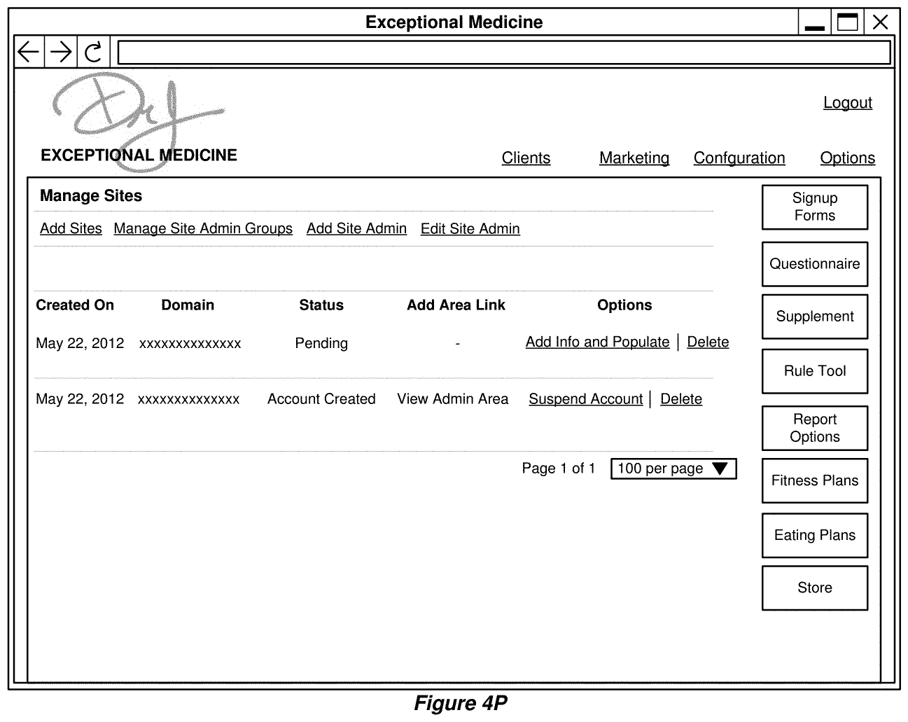

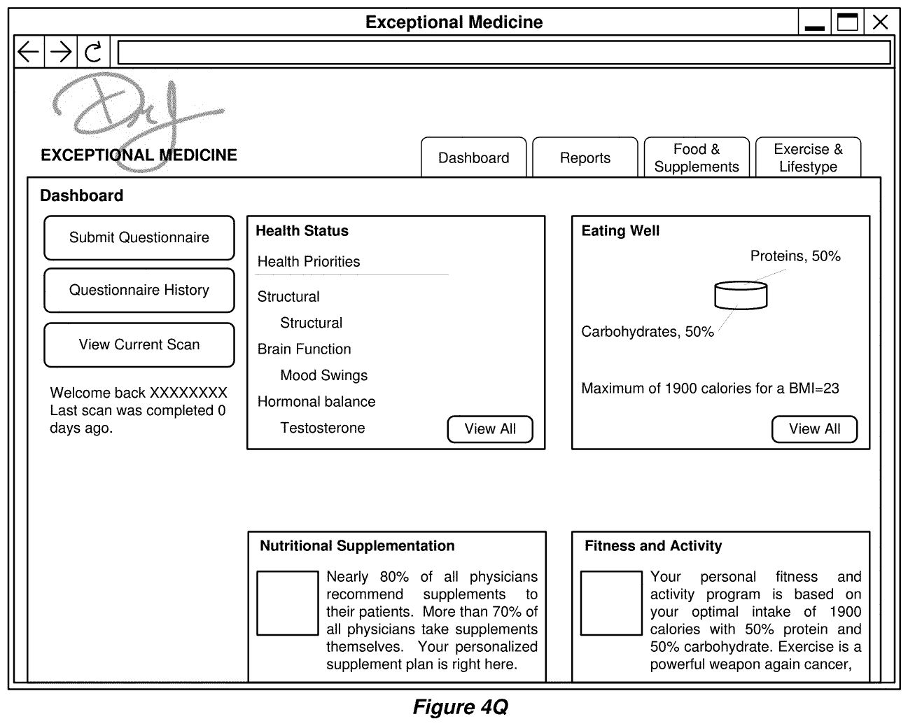

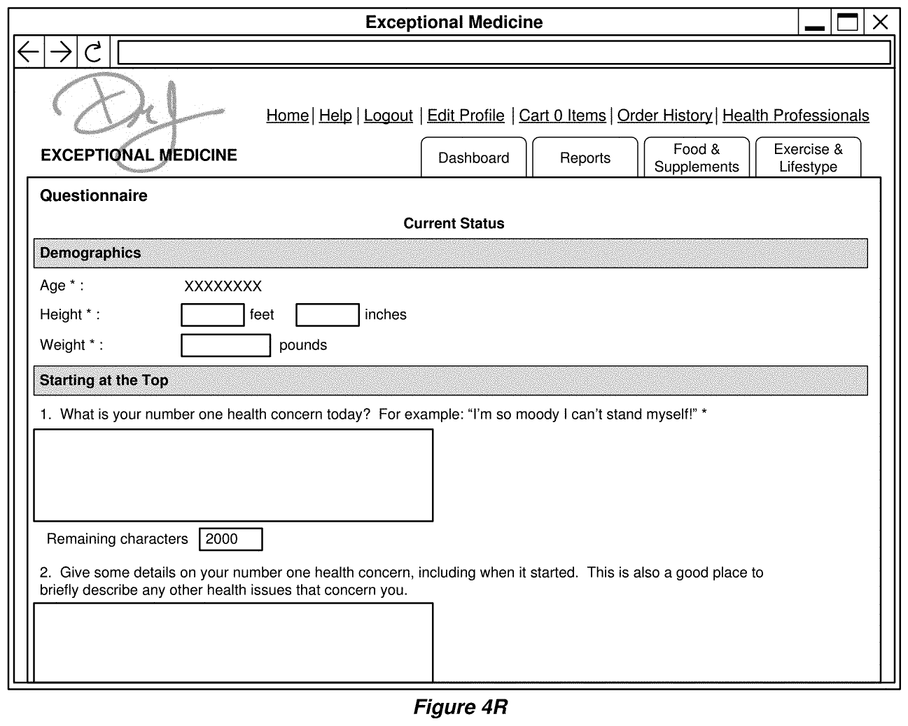

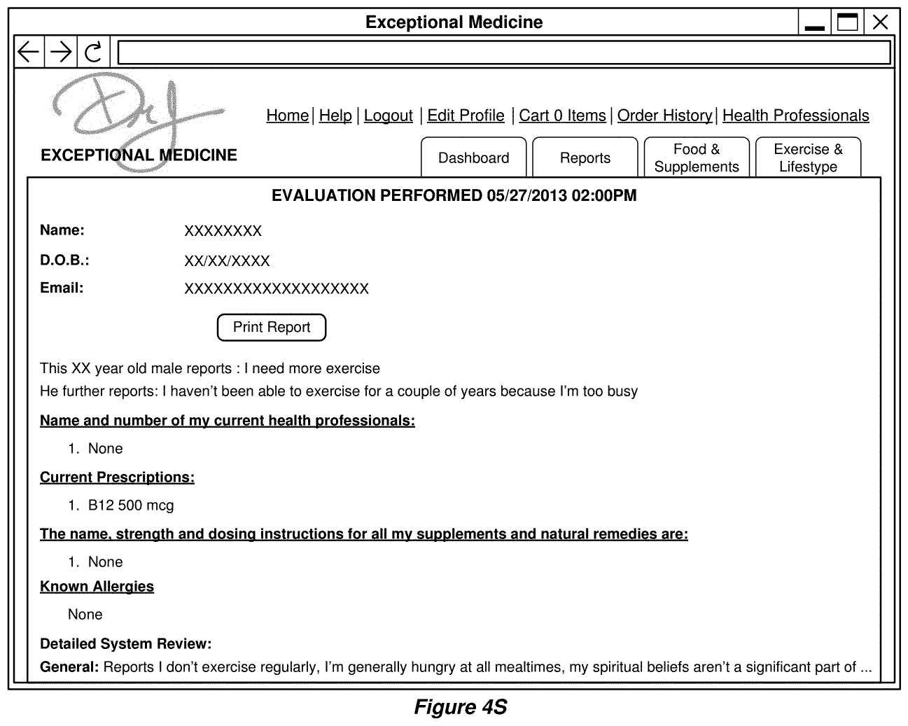

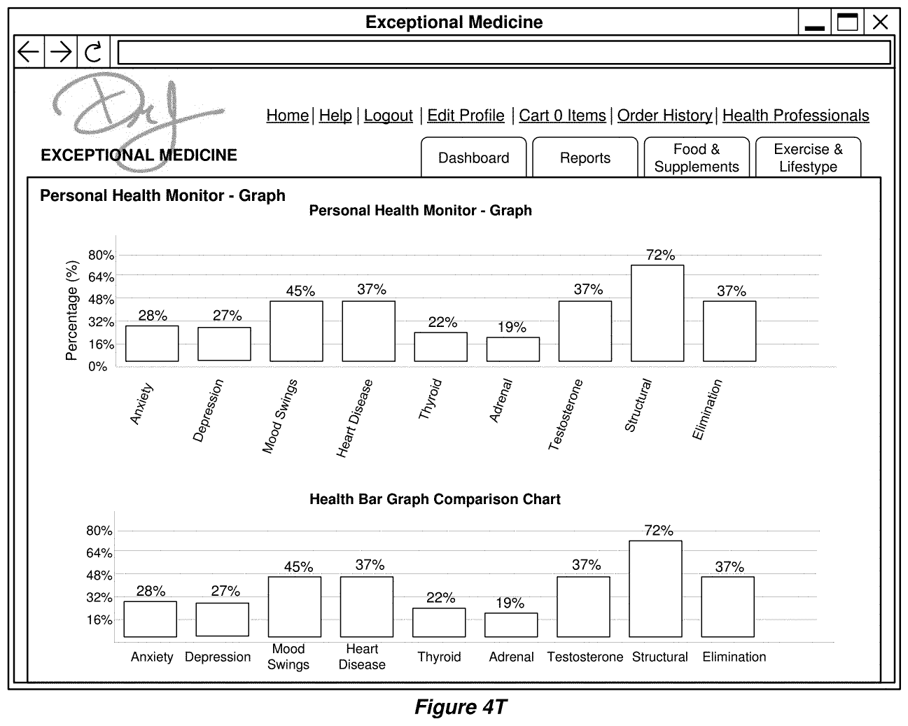

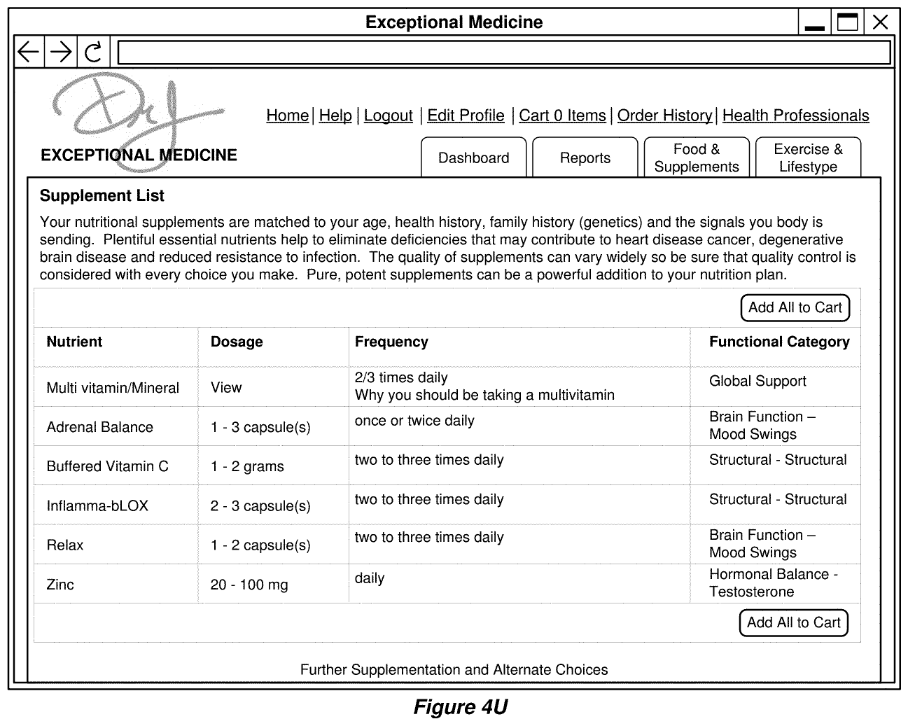

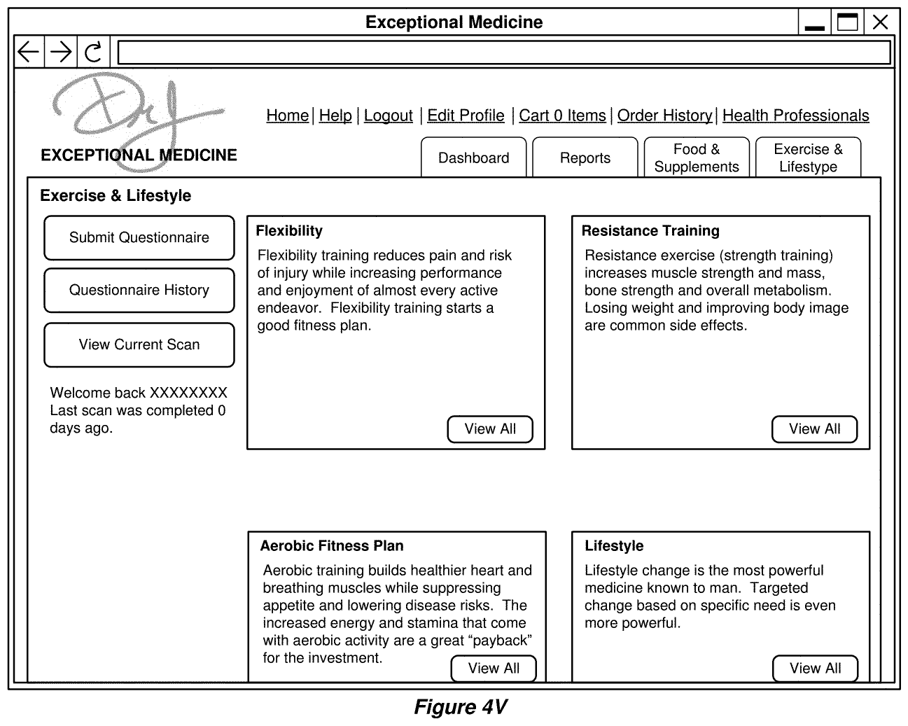

FIGS. 4A-4V illustrate an exemplary user interface and computer program for analyzing and managing fitness and nutritional wellness in accordance with an embodiment of the present invention. It is understood that other user interfaces are possible within the scope of the invention. According to one embodiment, when an administrator logs into the system 100, the administrator is presented with a graphical user interface similar to the graphical user interface shown in FIGS. 4A-4P that allows the administrator to configure the system 100. The menu options include, without limitation, “Clients”, “Marketing”, “Configuration”, “Options”, “Signup Form”, “Questionnaire”, “Supplement”, “Rule Tool”, “Report Options”, “Fitness Plans”, and “Eating Plans”.

If the administrator selects “Signup Forms”, the administrator can, without limitation, manage patient signup forms and coupons.

If the administrator selects “Questionnaire”, the administrator is presented with a graphical user interface as shown in FIG. 4A. The questionnaire is separated into different health and wellness sections, including without limitation “Current Status”, “General”, “Mind and Mood”, “Circulation and Breathing”, “Digestion and Elimination”, “Body Chemistry”, “Lifestyle and Social History”, “Structural” and “At Last”. New sections may be added using this graphical user interface. The sections may be edited by selecting “Manage Questions” and using a graphical user interface similar to one shown in FIGS. 4B-4D. Using the graphical user interface, the administrator can, without limitation, modify the question text, section, category, order, and report options.

If the administrator selects “Rule Tool”, the administrator is presented with a graphical user interface similar to one shown in FIGS. 4E-4G. Using the graphical user interface, the administrator can, without limitation, modify the rule description and the numerical correlation score associated with each question.

If the administrator selects “Supplements”, the administrator is presented with a graphical user interface similar to one shown in FIG. 4H. Using the graphical user interface, the administrator can, without limitation, modify supplement name, notes, warning, source, dosage, frequency, age group and gender information.

If the administrator selects “Report Options”, the administrator is presented with a graphical user interface similar to one shown in FIGS. 4I-4K. Using the graphical user interface, the administrator can, without limitation, add or modify report option categories, assign report options, and assign product to questions. Report option categories include without limitation, “General”, “HEENT”, “SKIN”, “CARDIOPULMONARY”, “DIGESTIVE”, “ENDOCRINE”, “IMMUNE”, “STRUCTURAL”, “GENITOURINARY” and “NEUROPSYCH”.

If the administrator selects “Fitness Plans”, the administrator is presented with a graphical user interface similar to one shown in FIG. 4L. Using the graphical user interface, the administrator can, without limitation, add or manage fitness plans, and exercises.

If the administrator selects “Eating Plans”, the administrator is presented with a graphical user interface similar to one shown in FIG. 4M. Using the graphical user interface, the administrator can, without limitation, add or manage eating plans, food type, food category and food.

If the administrator selects “Store”, the administrator is presented with a graphical user interface similar to one shown in FIG. 4N. Using the graphical user interface, the administrator can, without limitation, add or manage store products and shipping options.

If the administrator selects “Options”, the administrator is presented with a graphical user interface similar to one shown in FIG. 40. The menu options include, without limitation, “Brand Site”, “Store Options”, “Add Admin”, “Manage Sites”, “Update Standard Zip Files”, “Autoresponders”, and “Change Password”. If the administrator selects “Manage Sites”, the administrator is presented with a graphical user interface similar to the one shown in FIG. 4P. Using this graphical user interface, the administrator adds or manages sites and site administration.

According to one embodiment, when a patient logs into the system 100, the patient is presented with a graphical user interface similar to the graphical user interface shown in FIGS. 4Q-4U that allows the patient to access and improve their health and wellness. The menu options include, without limitation, “Dashboard”, “Reports”, “Food & Supplements”, and “Exercise & Lifestyle”.

If the patient selects “Dashboard”, the patient is presented with a graphical user interface similar to one shown in FIG. 4Q. The patient can, without limitation, submit and view questionnaires, and view current status. The patient is also provided information regarding the patient's health status, and provided recommendations regarding eating, nutritional supplements and fitness & activity.

If the patient selects “Submit Questionnaire”, the patient is presented with a graphical user interface similar to one shown in FIG. 4R. The patient can respond to the questions that have been predefined by the administrator using this user interface. Based on the responses, the patient may be provided with one or more reports or graphs using the graphical user interface similar to the graphical user interface shown in FIGS. 4S-4T. These reports and charts allow a patient to make improvements in their health and wellness.

If the patient selects “Food & Supplements”, the patient is presented with a graphical user interface similar to one shown in FIG. 4U. This user interface provides the patient with nutritional supplement recommendations based on their responses to the questions and the predefined correlation rules defined by the administrator.

If the patient selects “Exercise & Lifestyle”, the patient is presented with a graphical user interface similar to one shown in FIG. 4V. This user interface provides the patient with exercise and lifestyle recommendations based on their responses to the questions and the predefined correlation rules defined by the administrator.

It will be apparent to those skilled in the art that various modifications and variations can be made in the individualized health evaluation system and method of the present invention without departing form the spirit or scope of the invention. Thus, it is intended that the present invention cover the modifications and variations of the invention provided they come within the scope of the appended claims and their equivalents.

The present invention includes a computer program which may be hosted on a storage medium or other computer readable medium and includes instructions which perform the processes set forth herein. The storage medium or other computer readable medium can include, but is not limited to, any type of disk including floppy disks, optical disks, CD-ROMs, magneto-optical disks, ROMs, RAMs, EPROMs, EEPROMs, flash memory, magnetic or optical cards, or any type of media suitable for storing electronic instructions.

Obviously, many other modifications and variations of the present invention are possible in light of the above teachings. The specific embodiments discussed herein are merely illustrative, and are not meant to limit the scope of the present invention in any manner. It is therefore to be understood that within the scope of the disclosed concept, the invention may be practiced otherwise then as specifically described.

1. A system for analyzing and managing fitness and nutritional wellness, the system comprising:
a plurality of remote computers;
a central server;
a network interface in communication with the central server and the plurality of remote computers over a network, the network interface being configured to receive patient information;
a shared database in communication with the central server;
wherein the central server is configured to:
receive configuration and setup information, wherein the configuration and setup information includes configuring predefined fitness and wellness questions derived from validated questionnaires in a plurality of subspecialty fields, and predefined correlation rules associated with the fitness and wellness questions, and wherein predefined correlation rules include one or more predefined thresholds;
receive and store patient information in the database, wherein the patient information includes responses to the predefined fitness and wellness questions;
correlate the responses based on the predefined correlation rules to a plurality of functional scores;
create a health and wellness graphical representation of a patient based on the functional scores; and
display the health and wellness graphical representation within the predefined thresholds to the patient.

2. The computer program of claim 1, wherein the central server is further configured to maintain historical patient information in the database and display the historical patient information as a graphical representation.

3. The computer program of claim 1, wherein the correlation rules include assigning correlation scores to the predefined fitness and wellness questions.

4. The computer program of claim 1, wherein the predefined threshold is a range and fitness scores outside the range are ignored.

5. A computer program product embodied on a non-transitory computer readable medium for analyzing and managing fitness and nutritional wellness, wherein the computer program is implemented by one or more processors executing processor instructions, the computer program product comprising:
a first computer code for receiving configuration and setup information, wherein the configuration and setup information includes configuring predefined fitness and wellness questions, and predefined correlation rules associated with the fitness and wellness questions, and wherein predefined correlation rules include one or more predefined thresholds;
a second computer code for receiving and storing patient information in the database, wherein the patient information includes responses to the predefined fitness and wellness questions;
a third computer code for correlating the responses based on the predefined correlation rules to a plurality of functional scores;
a fourth computer code for creating a health and wellness graphical representation of a patient based on the functional scores; and
a fifth computer code for displaying the health and wellness graphical representation within the predefined thresholds to the patient.
